(19) United States
(12) Patent Application Publication
(10) Pub. No.: US 2024/0382633 A1
(43) Pub. Date: Nov. 21, 2024

(54) RADIOPHARMACEUTICALS, METHODS FOR THE PRODUCTION THEREOF, AND USES IN TREATMENT, DIAGNOSIS AND IMAGING DISEASES

(71) Applicant: Clarity Pharmaceuticals Ltd, Eveleigh (AU)

(72) Inventors: Lachlan Eion MCINNES, Eveleigh (AU); Paul Stephen DONNELLY, Melbourne (AU); Michael David IRONSIDE, Eveleigh (AU)

(21) Appl. No.: 18/684,582

(22) PCT Filed: Aug. 16, 2022
(86) PCT No.: PCT/AU2022/050905

§ 371 Date: Feb. 16, 2024

(30) Foreign Application Priority Data
Aug. 17, 2021 (AU) 2021902553

(51) Int. Cl. A61K 51/08; A61K 51/12; C07B 59/00

(52) U.S. Cl. CPC A61K 51/088; A61K 51/121; C07B 59/008; A61K 2121/00; A61K 2123/00; C07B 2200/05

(57) ABSTRACT

The present invention relates to compounds that can complex a radionuclide and formulations and kits comprising compounds that can complex a radionuclide. The compounds, formulations and kits are of use in radiotherapy and diagnostic imaging.

### FIELD

The present invention relates to compounds that can complex a radionuclide and may be useful as radiopharmaceuticals for the treatment, diagnosis and imaging of diseases such as cancer. The present invention also relates to methods for the production of such compounds and their complexes.

### BACKGROUND

Compounds that are capable of coordinating a radionuclide or radioisotope and also binding to a specific site in vivo may be suitable for use as a radiopharmaceutical. Such agents may allow for the treatment, diagnosis and imaging of diseases such as cancer. In addition to the requirements that a compound coordinate a radionuclide and bind at the desired site, the compound should also show sufficient stability for administration to patients, little to no dissociation of the radionuclide after administration and minimal side effects to the patient.

Drawbacks of known compounds used as radiopharmaceuticals include limited physiological stability, limited selectivity for a particular target and weak binding at the desired target. With respect to the radionuclide, dissociation of the radionuclide in vivo may lead to healthy tissue being exposed to radiation, which is undesirable. While some known metal chelators are capable of coordinating a radionuclide in vitro, administration to a patient under physiological conditions may lead to unwanted dissociation of the radionuclide or transchelation, where the radionuclide is transferred to another species capable of coordinating a metal ion.

While there are known processes to prepare compounds that may be suitable for use as radiopharmaceuticals, these processes often include coupling reactions under conditions that are incompatible with various parts of the intended compound. For example, where a particular functional group or moiety is known to bind a target site, unwanted modification of this group due to side reactions during preparation of the compound is undesirable and leads to an inactive compound.

There exists a need for compounds having the required binding affinity for targets in vivo, and the sufficient stability to bind at the desired target and deliver a radionuclide for the purposes of therapy, diagnosis or imaging. There also exists a need for processes to provide such compounds.

### SUMMARY OF THE INVENTION

According to a first aspect, the present invention provides a compound of Formula (I), or a salt, complex, isomer, solvate, prodrug or protected form thereof:

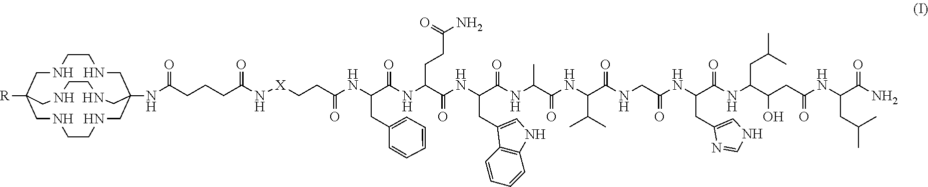

wherein:
X is

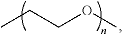

where n is an integer from 1 to 10;
R is a group selected from the group consisting of H, OH, halogen, cyano, NO₂, NH₂, optionally substituted C₁-C₁₂ alkyl, optionally substituted amino, optionally substituted amide, optionally substituted aryl and a group of the formula (A):

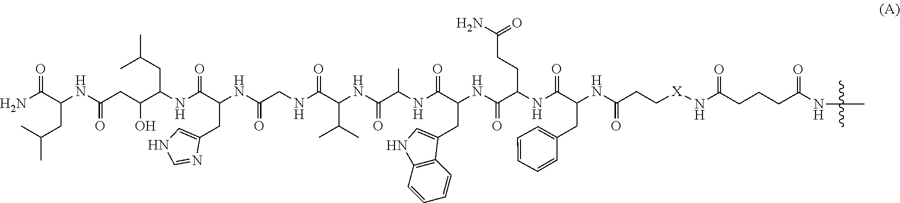

In an embodiment of the first aspect, R is a group of the formula (A):

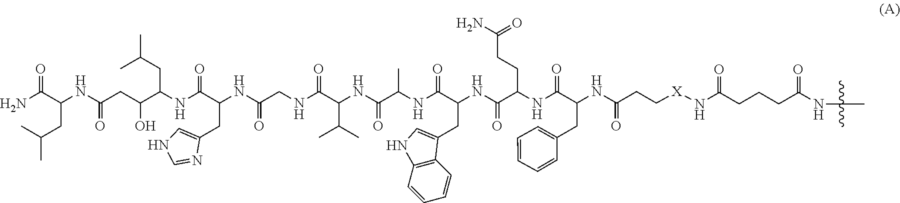

wherein X is as defined above, such as with the below stereochemistry:

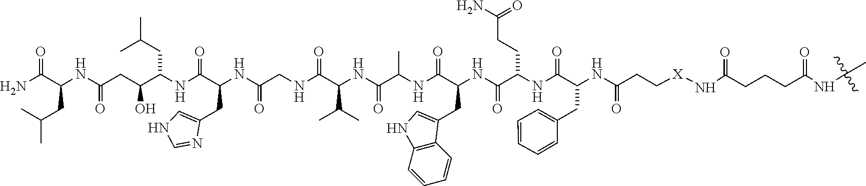

In an embodiment of the first aspect, X is

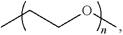

wherein n is an integer from 1 to 10.

In a further embodiment, X is

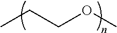

and n is 4.

In an embodiment of the first aspect, the compound of Formula (I), or the salt, complex, isomer, solvate, prodrug or protected form thereof, has the following structure of Formula (Ia):

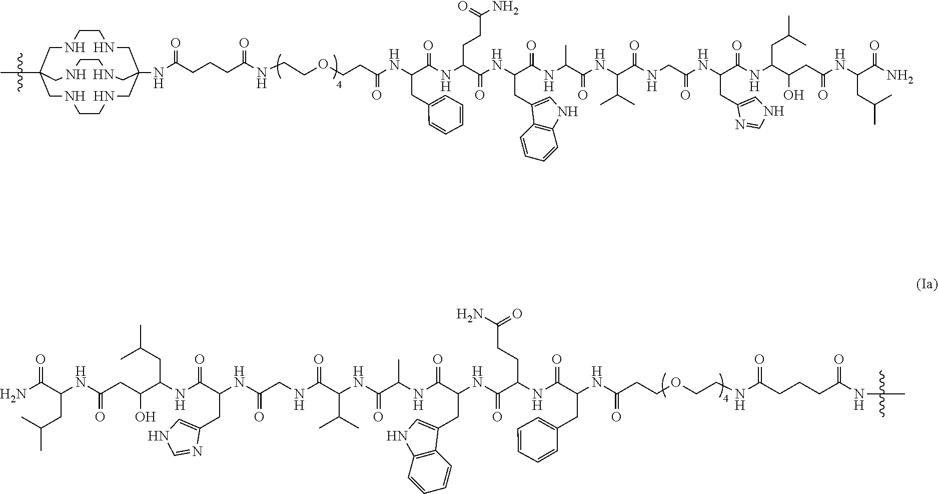

In an embodiment of the first aspect, the compound of Formula (I) or the salt, complex, isomer, solvate, prodrug or protected form thereof is coordinated with a metal ion.

In a further embodiment, the compound of Formula (I) or the salt, complex, isomer, solvate, prodrug or protected form thereof is complexed with a radionuclide of Cu.

In a further embodiment, the compound of Formula (I), or the salt, complex, isomer, solvate, prodrug or protected form thereof is complexed with a radionuclide selected from the group consisting of ⁶⁰Cu, ⁶¹Cu, ⁶²Cu, ⁶⁴Cu and ⁶⁷Cu.

In a further embodiment, the compound of Formula (I), or the salt, complex, isomer, solvate, prodrug or protected form thereof is complexed with radionuclides of cobalt.

In a further embodiment, the compound of Formula (I), or the salt, complex, isomer, solvate, prodrug or protected form thereof is complexed with a radionuclide selected from a group consisting of ⁵⁵Co, ⁵⁷Co and ^{58m}Co.

In a further embodiment, the compound of Formula (I), or the salt, complex, isomer, solvate, prodrug or protected form thereof is complexed with In.

In a further embodiment, the compound of Formula (I), or the salt, complex, isomer, solvate, prodrug or protected form thereof is complexed with the radionuclide ¹¹¹In.

In a further embodiment, the compound of Formula (I), or the salt, complex, isomer, solvate, prodrug or protected form thereof is complexed with Sc.

In a further embodiment, the compound of Formula (I), or the salt, complex, isomer, solvate, prodrug or protected form thereof is complexed with a radionuclide selected from a group consisting of ⁴³Sc, ⁴⁴Sc and ⁴⁷Sc.

In a further embodiment, the compound of Formula (I), or the salt, complex, isomer, solvate, prodrug or protected form thereof is complexed with a radionuclide selected from ⁶⁸Ga or ⁶⁷Ga, or ¹⁸⁸Re or ¹⁸⁶Re.

In a further embodiment, the compound of Formula (I), or the salt, complex, isomer, solvate, prodrug or protected form thereof is complexed with a radionuclide selected from ⁶²Mn and ⁴⁵Ti.

According to a second aspect, the present invention provides a composition comprising a compound according to the first aspect, and one or more pharmaceutically acceptable excipients.

According to a third aspect, the present invention provides a process for producing a compound of Formula (I) or a salt, complex, isomer, solvate, prodrug or protected form thereof:

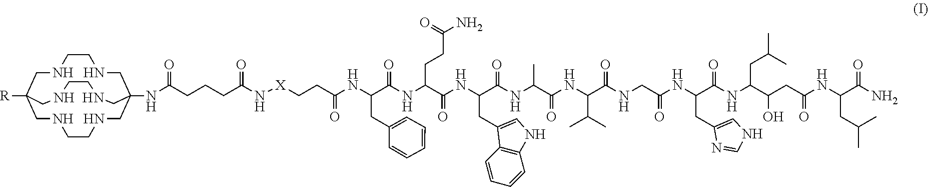

wherein X is as defined in claim 1 and R is a group of the Formula (A):

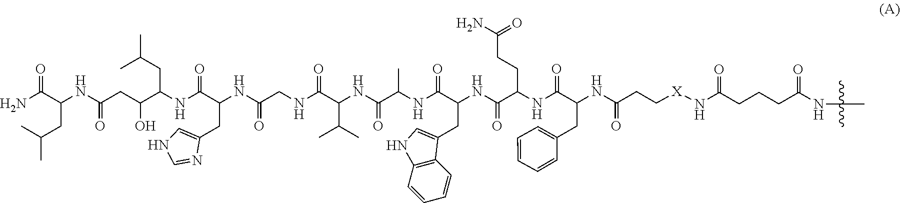

the method comprising the step of coupling a compound of the Formula (II), or a salt, complex, isomer or solvate thereof,

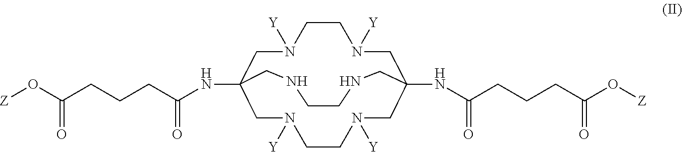

wherein Y is a nitrogen-protecting group and Z is an oxygen-protecting group;
with a compound of Formula (III) or a salt thereof,

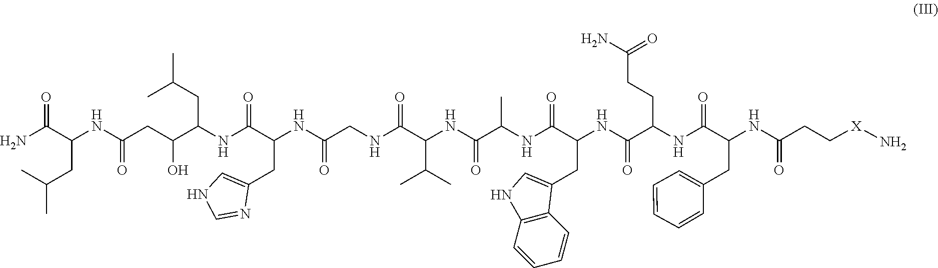

for a time and under conditions to provide a compound of Formula (I).

In an embodiment of the third aspect, the process is performed under microwave conditions.

According to a fourth aspect, the present invention provides a method of treating a cancer in a subject, the method comprising administering to a subject in need thereof a compound as defined in the first aspect, wherein the compound is complexed with a radionuclide.

According to a fifth aspect, the present invention provides a method of radioimaging a subject, the method comprising administering to a subject in need thereof a compound as defined in the first aspect, wherein the compound is complexed with a radionuclide.

According to a sixth aspect, the present invention provides an aqueous formulation comprising a compound of Formula (I), or a salt, isomer, solvate, prodrug or protected form thereof, complexed with a radionuclide:

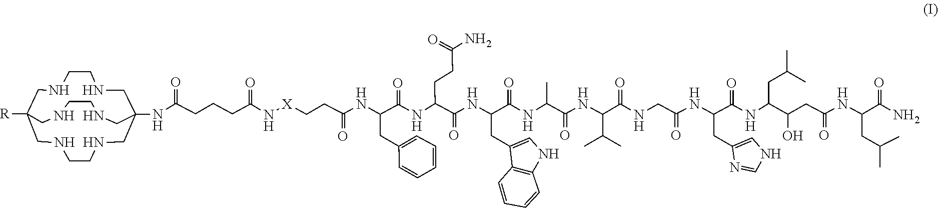

wherein:
X is

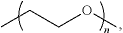

where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl and a group of the formula (A):

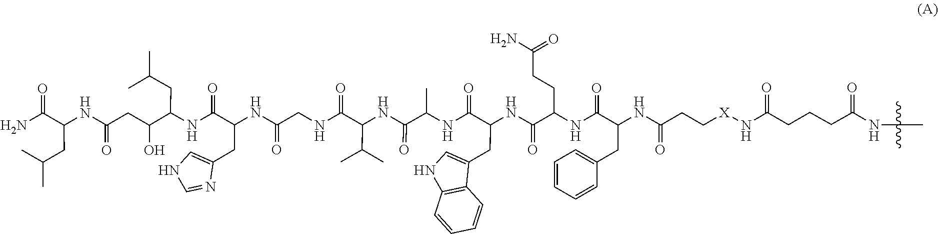

the formulation comprising a buffer and one or more excipients.

In an embodiment of the sixth aspect, the compound of Formula (I), or the salt, isomer, solvate, prodrug or protected form thereof, is of the Formula (Ib):

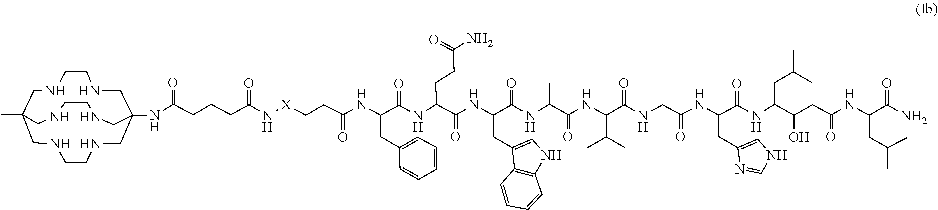

wherein:

X is

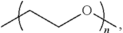

where n is 4; and
the formulation comprising sodium phosphate buffer, about 0.01% to about 0.1% (w/v) gentisic acid, or a salt thereof, about 3.0% to about 9.0% (w/v) ascorbic acid, or a salt thereof, and about 1% to about 7% (v/v) ethanol.

According to a seventh aspect, the present invention provides a method of treating a cancer in a subject, the method comprising administering to a subject in need thereof the aqueous formulation according to the sixth aspect.

According to an eighth aspect, the present invention provides a method of radioimaging a subject, the method comprising administering to a subject in need thereof the aqueous formulation according to the sixth aspect.

### BRIEF DESCRIPTION OF THE FIGURES

FIG. 1: RadioHPLC trace of [⁶⁴Cu]CuSar-bombesin₂.

FIG. 2: Representative radio-TLC trace of purified peptide showing successful labelling with ⁶⁴Cu. Two radio-TLCs were run for each sample, without 50 mM EDTA (left) and with 50 mM EDTA (right). Without EDTA, both the radiolabelled peptide and unbound radioisotope are represented at ˜50 mm. With EDTA, the radiolabelled peptide is represented at ˜120 mm, while the unbound radioisotope is represented at ˜160 mm. Both radio-TLCs show that there is negligible unbound ⁶⁴Cu.

FIG. 3: Axial, coronal and sagittal PET-CT projections of a representative mouse injected with [⁶⁴Cu]Formula (Ia) at 1 hour (A), 4 hours (B) and 24 hours (C).

FIG. 4: Axial, coronal and sagittal PET-CT projections of a representative mouse injected with [⁶⁴Cu]Formula (Ib) at 1 hour (A), 4 hours (B) and 24 hours (C).

FIG. 5: Biodistribution of [⁶⁴Cu]Formula (Ia) (A) and [⁶⁴Cu]Formula (Ib) (B) at 1, 4 and 24 hours post-injection as determined by ROI analysis of PET images.

FIG. 6: Stability profile of [⁶⁴Cu]Formula (Ib) in an aqueous formulation over 48 hours.

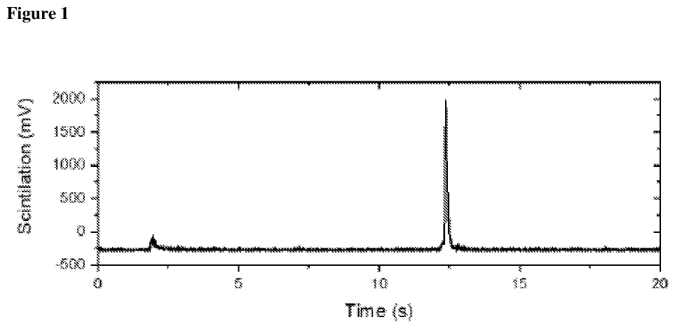

### DETAILED DESCRIPTION

The present invention provides compounds of Formula (I), salts, complexes, isomers, solvates, prodrugs and protected forms thereof:

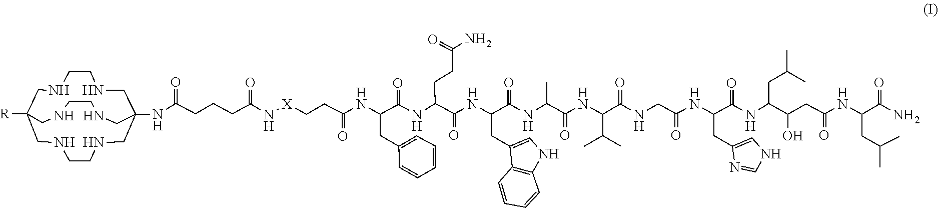

wherein X is

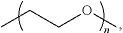

where n is an integer from 1 to 10;
R is a group selected from the group consisting of H, OH, halogen, cyano, NO₂, NH₂, optionally substituted C₁-C₁₂ alkyl, optionally substituted amino, optionally substituted amide, optionally substituted aryl and a group of the formula (A):

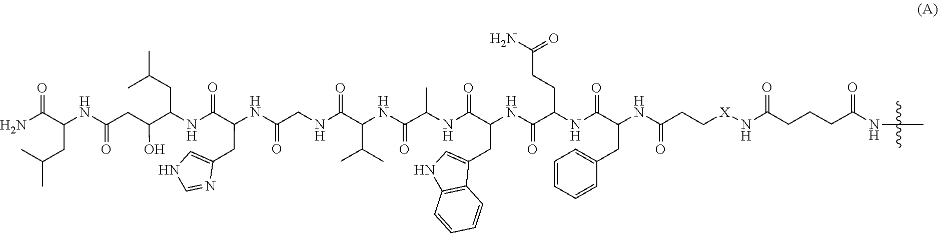

wherein X is a as defined above.

The compounds of Formula (I), or the salts, complexes, isomers, solvates, prodrugs or protected forms thereof contain a peptide, where the peptide has the sequence D-Phe-Gln-Trp-Ala-Val-Gly-His-Sta-Leu-NH₂ and has the following structure:

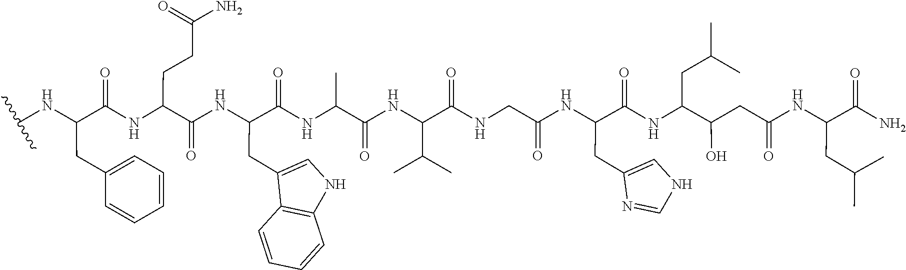 [SMILES: *NC(Cc1ccccc1)C(=O)NC(CCC(N)=O)C(=O)NC(Cc1c[nH]c2ccccc12)C(=O)NC(C)C(=O)NC(C(=O)NCC(=O)NC(Cc1c[nH]cn1)C(=O)NC(CC(C)C)C(O)CC(=O)NC(CC(C)C)C(N)=O)C(C)C]

The above peptide fragment is related to the family of bombesin receptor peptides that show antagonist (or agonist) activity at the gastrin-releasing peptide (GRP) receptor. The GRP receptor is known to be overexpressed on the membrane of various cancers and may be a target for diagnostic or therapeutic purposes. Compounds containing the bombesin-like peptide as depicted herein may bind to sites expressing the GRP receptor and where a suitable radionuclide is also delivered as part of the compound, a diagnostic or therapeutic effect may be provided locally. The amino acids of the bombesin-like peptide as used herein may have a specific stereochemistry, as depicted below:

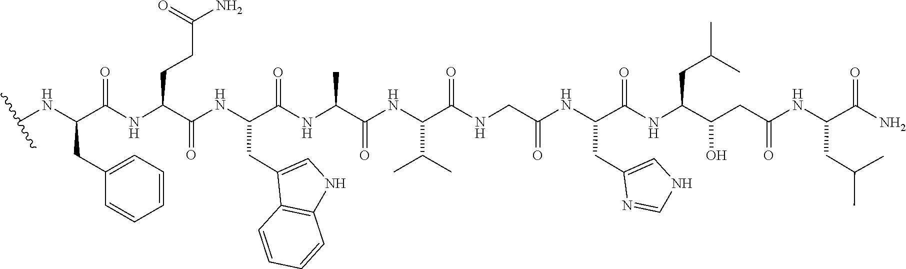

The compounds of Formula (I) or the salts, complexes, isomers, solvates, prodrugs or protected forms thereof also contain a nitrogen-containing macrocycle, which is capable of chelating metal ions. The macrocycle of Formula (I) is a 3,6,10,13,16,19-hexaazabicyclo[6.6.0]icosane and may be referred to as a “sarcophagine”. The sarcophagines of Formula (I), or the salts, complexes, isomers, solvates, prodrugs and protected forms thereof contain six nitrogen atoms, where one or more of the nitrogen atoms may be protected with a suitable protecting group.

The compounds of Formula (I), or the salts, complexes, isomers, solvates, prodrugs or protected forms thereof contain a sarcophagine and a bombesin-like peptide, where the peptide is bound to terminal position of the sarcophagine via a linker group. As depicted herein, the linker group comprises a propylamide group bound directly to the terminal position of the sarcophagine. The propylamide group is then attached to a linker comprising a polyethylene glycol (PEG) group, having between 1 and 10 repeat units. The PEG group has the following structure:

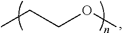

where n is an integer from 1 to 10.

The present inventors have found that compounds of Formula (I), or salts, complexes, isomers, solvates, prodrugs and protected forms thereof containing the combination of a sarcophagine and a bombesin-like peptide, or peptide that serves as either an agonist or antagonist for the gastrin-releasing peptide receptor, for instance, where the sarcophagine and the bombesin-like peptide are bound together via a propylamide group (adjacent to the sarcophagine) and the linker comprising the PEG group are capable of chelating a metal ion and binding to a target receptor. Without wishing to be bound by theory, the present inventors believe that it is the combination of the sarcophagine, bombesin-like peptide, propylamide group and the linker comprising a PEG group that provides the specific advantages that are observed and discussed below. Although the properties of the compounds of the present invention are as a result of each component of the compound, the present inventors believe that the presence of the linker comprising a PEG group to modify biodistribution, metabolism and excretion profiles, increases the overall biocompatibility of the compounds and may be responsible for the observed advantages.

In certain embodiments, the group R in the compound of Formula (I), or the salt, complex, isomer, solvate, prodrug or protected form thereof is selected from the group consisting of H, OH, halogen, cyano, NO₂, NH₂, optionally substituted C₁-C₁₂ alkyl, optionally substituted amino, optionally substituted C₁-C₁₂ amide, optionally substituted C₆-C₁₀ aryl and a group of the formula (A):

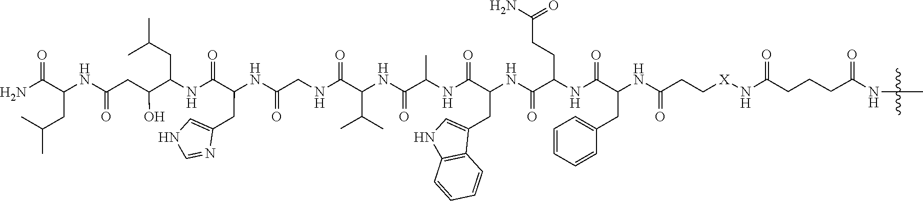

wherein X is as defined above.

In certain embodiments, R is a group of the formula (A) having the stereochemistry as defined below:

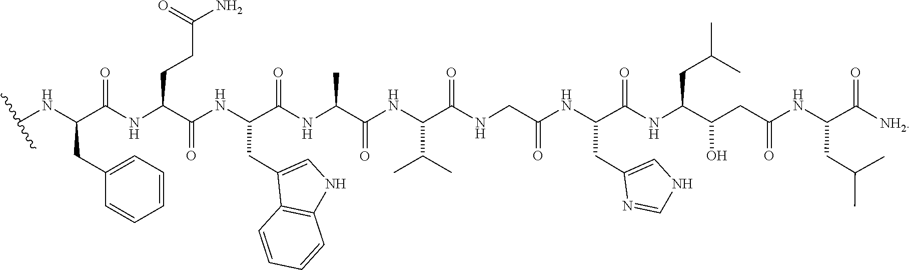

In certain embodiments, R is an optionally substituted C₁-C₁₂ alkyl group. In an embodiment, R is an optionally substituted C₁ alkyl group. In another embodiment, R is an optionally substituted methyl group. In another embodiment, R is an unsubstituted C₁-C₁₂ alkyl group. In another embodiment, R is an unsubstituted C₁ alkyl group. In another embodiment, R is an unsubstituted methyl group.

In certain embodiments, the compound of Formula (I), or the salt, complex, isomer, solvate, prodrug or protected form thereof has the following structure:

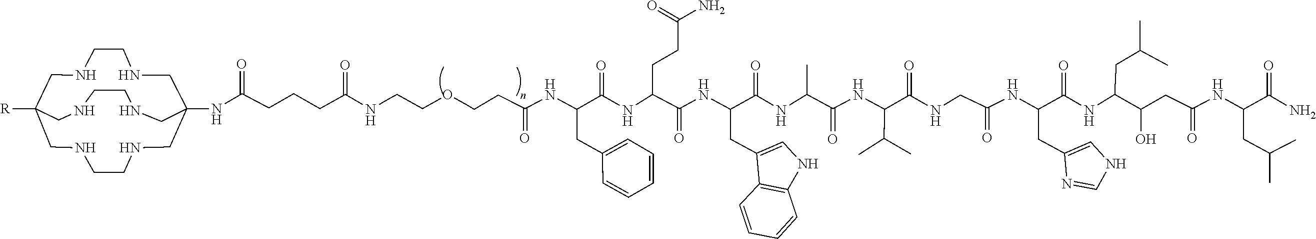

wherein n is an integer from 1 to 10.

In a specific embodiment, the compound of Formula (I), or the salt, complex, isomer, solvate, prodrug or protected form thereof has the following structure:

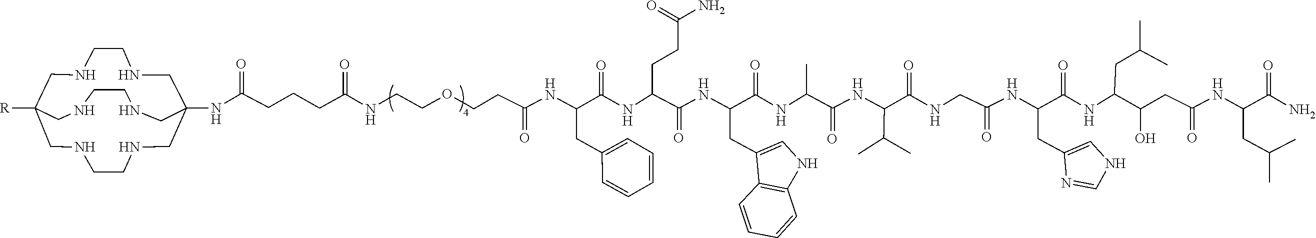

In certain embodiments, R is an optionally substituted C₁-C₁₂ amide group. In an embodiment, R is an optionally substituted C₁ amide group. In an embodiment, R is a C₁ amide group that is further substituted by one or more groups.

In certain embodiments, R is a group of the formula (A):

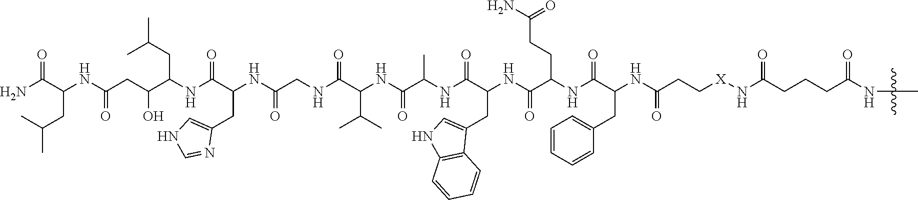

wherein X is as defined above.

In an embodiment, R is a group of the formula (A) and X is a group of the formula

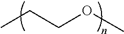

wherein n is an integer from 1 to 10.

In an embodiment, R is a group of the formula (A), X is a group of the formula

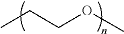

and n is 4.

In certain embodiments, the compound of Formula (I) has the following structure of Formula (Ia):

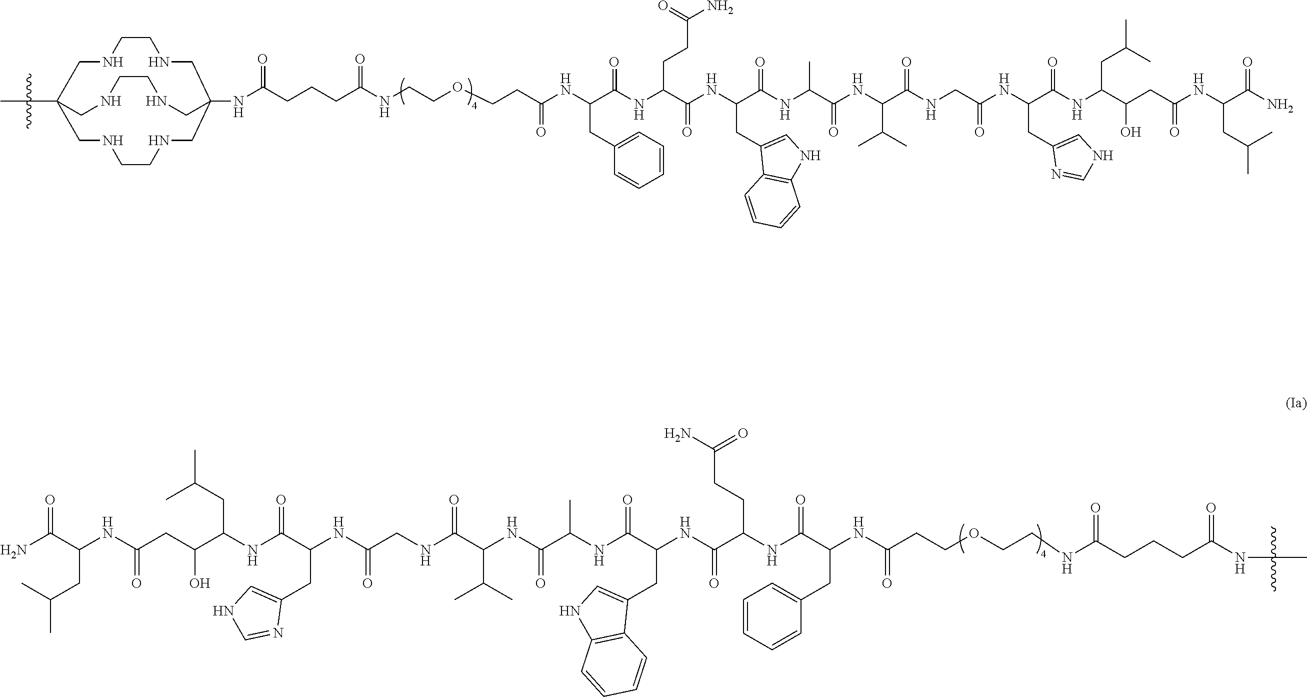

In other embodiments, the compound of Formula (I), or the salt, complex, isomer, solvate, prodrug or protected form thereof has the structure of Formula (Ia), where the stereochemistry is defined as below:

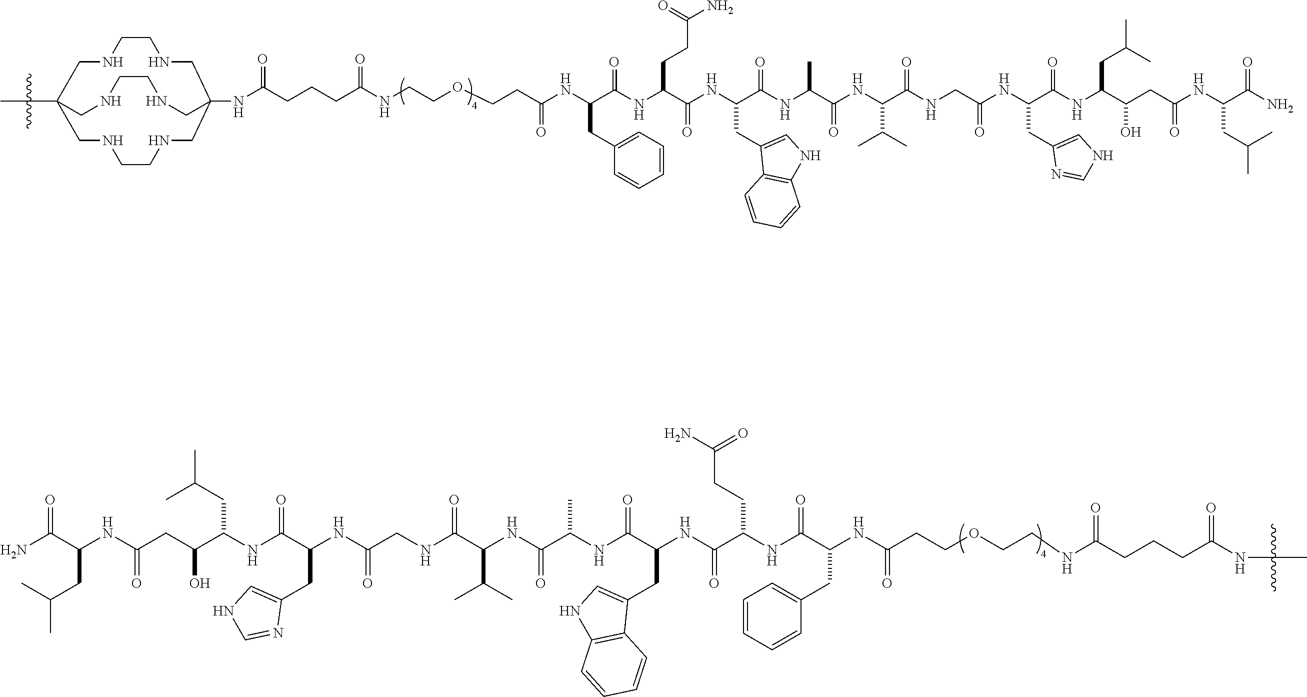

The compound of Formula (Ia), or the salt, complex, isomer, solvate, prodrug or protected form thereof has two bombesin-like peptides installed at opposite ends of the compounds. The compounds having the structure of Formula (Ia) may have better binding affinity in vivo when compared to the analogous substituted sarcophagine having a single bombesin-like peptide. The compound of Formula (Ia), or the salt, complex, isomer, solvate, prodrug or protected form thereof may also have improved metabolic stability, better biodistribution, uptake and clearance in vivo, when compared to compounds having a single bombesin-like peptide. In relation to dosing of patients for the purposes of treatment of a cancer or for radioimaging, these properties may allow for single dosing of the compounds, rather than administration of multiple doses, or multiple dosing at lower concentrations, in order to provide a therapeutic or effective dose in vivo. Alternatively, these properties may allow for multiple dosing at the same or higher concentrations for even better therapeutic efficacy.

As used herein, the term “alkyl” refers to a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like.

As used herein, the term “amide” refers to a functional group consisting of a carbonyl group attached to a nitrogen atom. Therefore, the term “optionally substituted amide” refers to an amide functional group that bears further substitution.

As used herein, the term “aryl” refers to a group or part of a group that denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. Typically an aryl group is a C₆-C₁₈ aryl group.

As used herein, the term “cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₉ cycloalkyl group.

As used herein, the term “halogen” represents chlorine, fluorine, bromine or iodine.

As used herein, the term “heteroalkyl” refers to a straight- or branched-chain alkyl group preferably having from 2 to 12 carbons, more preferably 2 to 6 carbons in the chain, in which one or more of the carbon atoms (and any associated hydrogen atoms) are each independently replaced by a heteroatomic group selected from S, O, P and NR′ where R′ is selected from the group consisting of H, optionally substituted C₁-C₁₂alkyl, optionally substituted C₃-C₁₂cycloalkyl, optionally substituted C₆-C₁₈aryl, and optionally substituted C₁-C₁₈heteroaryl. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl

As used herein, the term “heteroaryl” either alone or as part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl, 1-, 2-, or 3-indolyl, and 2-, or 3-thienyl. A heteroaryl group is typically a C₁-C₁₈ heteroaryl group.

As used herein, the term “C₁-C₁₂ alkylene” refers to a bivalent straight or branched chain aliphatic hydrocarbon group, where the group has 1 to 12 carbon atoms in the chain.

As used herein, the term “optionally substituted” used in connection with a particular group indicates that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{a}, —C(═O)OR^{a}, C(═O)NR^{a}R^{b}, C(═NOH)R^{a}, C(═NR^{a})NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NR^{b})NR^{c}R^{d}, NR^{a}SO₂R^{b}, —SR^{a}, SO₂NR^{a}R^{b}, —OR^{a}, OC(═O)NR^{a}R^{b}, OC(═O)R^{a} and acyl, wherein R^{a}, R^{b}, R^{c} and R^{d} are each independently selected from the group consisting of H, C₁-C₁₂alkyl, C₁-C₁₂haloalkyl, C₂-C₁₂alkenyl, C₂-C₁₂alkynyl, C₂-C₁₀heteroalkyl, C₃-C₁₂cycloalkyl, C₃-C₁₂cycloalkenyl, C₂-C₁₂heterocycloalkyl, C₂-C₁₂heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments, each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

Examples of particularly suitable optional substituents include F, Cl, Br, I, CH₃, CH₂CH₃, OH, OCH₃, CF₃, OCF₃, NO₂, NH₂, COOH, COOCH₃ and CN.

As used herein, the term “salt” refers to acid addition salts and base addition salts of the compound, where the salt is prepared from an inorganic or organic acid, or an inorganic or organic base. In some embodiments, the salts of the compounds of the present invention may be pharmaceutically acceptable salts.

As used herein, the term “pharmaceutically acceptable salts” refers to salts that retain the desired biological activity of the above-identified compounds and may also be acid addition salts or base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

As used herein, the term “complex” refers to a compound that is then coordinated by a metal ion.

As used herein, the term “solvate” refers to a complex of the compound, where the complex may have variable stoichiometry formed by a solute and a solvent. Such solvents in the solvate should not interfere with the biological activity of the solute. Examples of suitable solvents may include water, ethanol or acetic acid. Methods of solvation of the compound are generally known in the art.

As used herein, the term “prodrug” refers to and includes derivatives that are converted in vivo to the compounds of the present invention. Such derivatives would readily occur to those skilled in the art, and include, for example, compounds containing a free hydroxyl group that is converted into an ester derivative, or containing a ring nitrogen atom that is converted to an N-oxide. Examples of ester derivatives include alkyl esters, phosphate esters and those formed from amino acids.

The compounds of Formula (I), or the salts, isomers, solvates, prodrugs or protected forms thereof may be coordinated with a metal ion via the nitrogen-containing macrocycle to form the corresponding complexes of Formula (I). In an embodiment, the compound of Formula (I) is coordinated with a metal ion.

In an embodiment, the metal ion is an ion of Cu, Tc, Gd, Ga, In, Co, Re, Fe, Au, Mg, Ca, Ag, Rh, Pt, Bi, Cr, W, Ni, V, Ir, Zn, Cd, Mn, Ru, Pd, Hg, Ti, Lu, Sc, Zr, Pb, Ac and Y.

In an embodiment, the metal ion is a radionuclide. In some embodiments, the metal ion is a radionuclide of a metal selected from the group consisting of Cu, Tc, Ga, Co, In, Fe, and Ti. The present compounds have been found to be particularly applicable useful in binding copper ions. In some embodiments, the metal ion is a radionuclide selected from the group consisting of ⁶⁰Cu, ⁶¹Cu, ⁶²Cu, ⁶⁴Cu and ⁶⁷Cu. In some embodiments, the radionuclide is ⁶⁰Cu. In some embodiments the radionuclide is ⁶¹Cu. In some embodiments, the radionuclide is ⁶²Cu. In some embodiments the radionuclide is ⁶⁴Cu. In some embodiments, the radionuclide is ⁶⁷Cu. The present compounds have also been found to be useful in binding cobalt ions. In some embodiments, the metal ion is a radionuclide of cobalt. In some embodiments, the radionuclide is ⁵⁵Co.

Where the metal ion is a radionuclide and the compound for Formula (I), or the salt, isomer, solvate, prodrug or protected form thereof is radiolabelled to form a complex, the complex may be administered for the purposes of radiotherapy or radioimaging. As discussed earlier, compounds (and subsequently, the radiolabelled complexes) of Formula (I) contain one or more bombesin-like peptides that are capable of binding GRP receptor, therefore the radiolabelled complexes of Formula (I), or the salts, isomers, solvates, prodrugs or protected forms thereof may be used for the radiotherapy or radioimaging of cancers that are associated with overexpression of the GRP receptor.

The present inventors have found that the compounds and complexes of Formula (I), or the salts, isomers, solvates, prodrugs or protected forms thereof containing a sarcophagine, a bombesin-like peptide, the propylamide linker and the linker comprising a PEG group shows affinity for the GRP receptor. The combination of each of these components in the compound of Formula (I) allow for administration of the corresponding complex containing a radionuclide, maintaining stability of the complex in vivo and accumulation of the complex at the intended target.

The compounds of the present invention and complexes thereof with a radionuclide may be used in methods of radioimaging, diagnosis or therapy.

Radioimaging of a cancer associated with overexpression of the GRP receptor in connection with the administration of a complex of Formula (I), or a salt, isomer, solvate, prodrug or protected form thereof, relies upon the selection of a suitable radionuclide. For example, where the intended use of a complex of Formula (I) is for the purposes of radioimaging, the selected radionuclide should have a sufficiently long half-life such that detection of radionuclide decay allows for images of a sufficient quality to be obtained. This also requires that the compound of Formula (I) itself, i.e. the ligand coordinating the radionuclide, be sufficiently stable with respect to radioactive decay. The present inventors have found that decomposition of a complex of Formula (I) by radiolysis (i.e. as a result of the radioactivity of the radionuclide) is minimized and that the complex of Formula (I) generally remains intact in this regard.

Radioimaging of a subject to which a radiolabeled compound of Formula (I), or a salt, isomer, solvate, prodrug or protected form thereof is administered may be by positron emission tomography (PET) or by single-photon emission computed tomography (SPECT). In an embodiment, the present invention provides a method for radioimaging a subject in need thereof, the method comprising administering a compound of Formula (I), or a salt, isomer, solvate, prodrug or protected form thereof complexed with a radionuclide. In an embodiment, the method comprises administering a compound of Formula (I), or a salt, isomer, solvate, prodrug or protected form thereof having the structure of Formula (Ia):

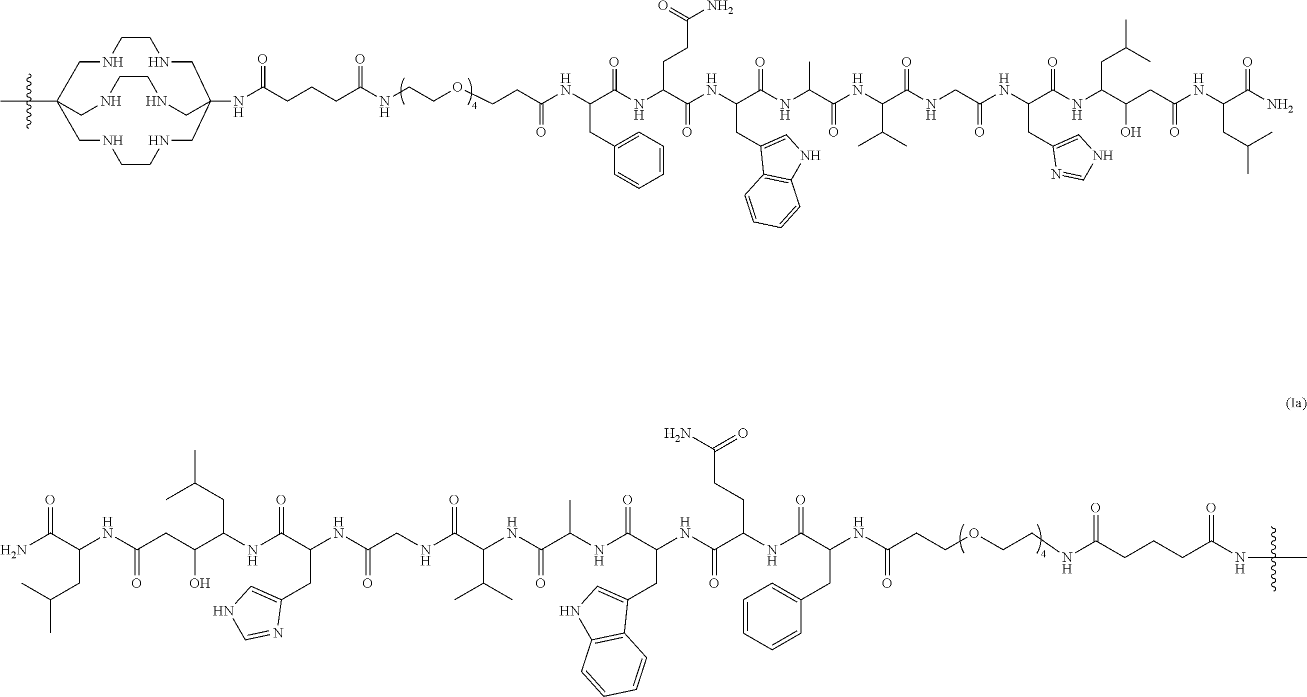

wherein the compound of Formula (Ia) is complexed with a radionuclide. In a further embodiment, the radionuclide is selected from the group consisting of ⁶⁰Cu, ⁶¹Cu, ⁶²Cu, ⁶⁴Cu and ⁶⁷Cu. In some embodiments the radionuclide is ⁶⁰Cu. In some embodiments the radionuclide is ⁶¹Cu. In some embodiments the radionuclide is ⁶²Cu. In some embodiments the radionuclide is ⁶⁴Cu. In some embodiments the radionuclide is ⁶⁷Cu.

In an embodiment, radioimaging of the subject after administration of the compound of Formula (I) complexed by a radionuclide is by PET. In another embodiment, radioimaging of the subject after administration of the compound of Formula (I) complexed by a radionuclide is by SPECT.

The compounds of the present invention complexed with a radionuclide may be administered to a subject in need thereof as a composition by a parenteral route. Administration by intravenous injection may be preferred. Alternatively, the formulations of the present invention may be given by intraarterial or other routes, for delivery into the systemic circulation. The subject to which the compound is administered is then placed into a PET (or SPECT) scanner and images showing the localisation of the complex, and subsequently location of any cancers or tumours, are obtained. This then allows for diagnosis and detection of a cancer or tumour.

The compounds of the present invention and complexes thereof with a radionuclide may be used in methods of treatment of diseases, such as cancers. When complexed with a suitable radionuclide, the complexes of the present invention may be administered to a subject in need thereof. The methods disclosed herein comprise administration of an effective amount of a radiolabeled compound of the present invention to a subject in need thereof. The compound contains a bombesin-like peptide, which binds at GRP receptors that are overexpressed at sites of various cancers. Given the abundance of such receptors are associated with a particular type of cancer, the accumulation of compounds of the present invention as detected by the radioactive decay of the radionuclide indicates the location of the cancer. The present inventors have found that compounds of the present invention show a particular affinity for the GRP receptor. Furthermore, the presence of both the propylamide linker and the linker comprising a PEG group contribute to provide a complex (when the compound is radiolabeled with a radionuclide) that is capable of administration to a subject and subsequent localization at sites overexpressing the GRP receptor. The compounds of the present invention also have the requisite stability with respect to the radionuclide. For example, the sarcophagine present in the compound is capable of chelating a radionuclide such that the radionuclide remains coordinated upon administration to a subject and subsequent binding at the target site. Since the radionuclide remains coordinated and localized to the target site due to binding of the compound as a whole, radiation damage at other sites (e.g. healthy tissue) is minimized.

In an embodiment, a method for the treatment of a cancer comprises administering a compound of Formula (I), or a salt, isomer, solvate, prodrug or protected form thereof complexed with a radionuclide. In an embodiment, the method comprises administering a compound of Formula (I), or a salt, isomer, solvate, prodrug or protected form thereof having the structure of Formula (Ia):

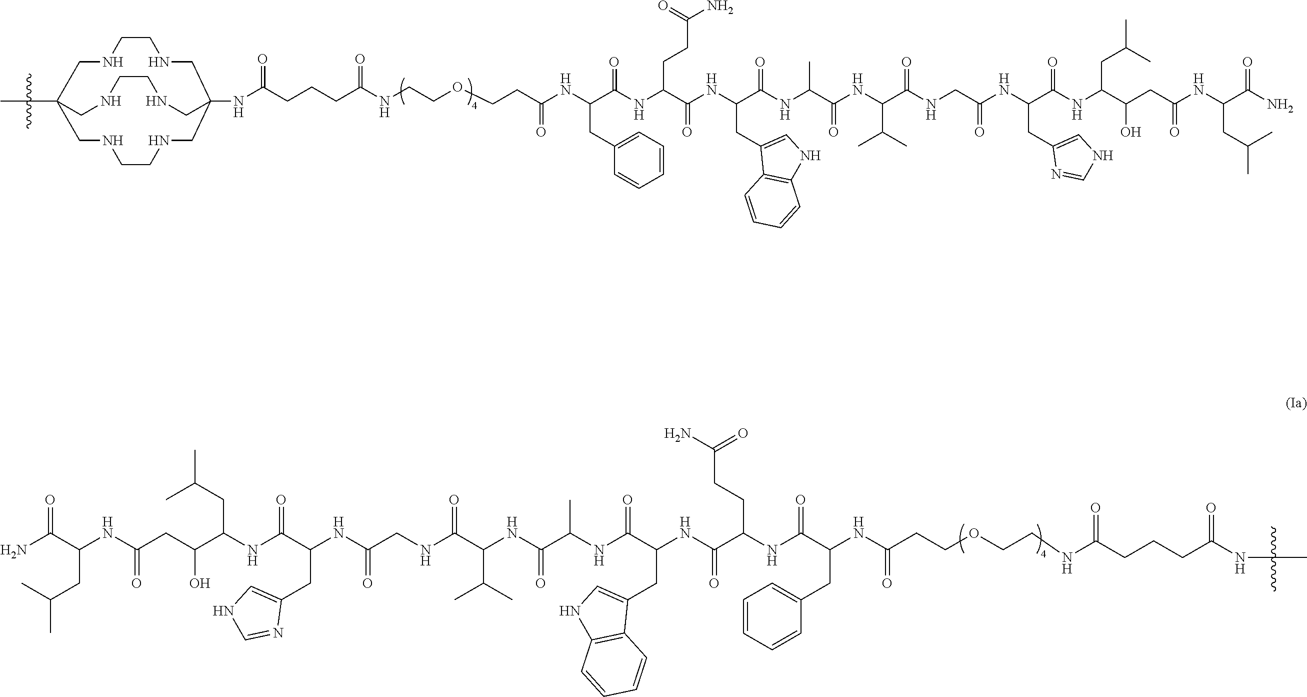

wherein the compound of Formula (Ia) is complexed with a radionuclide. In a further embodiment, the radionuclide is selected from the group consisting of ⁵⁵Co, ⁶⁰Cu, ⁶¹Cu, ⁶²Cu, ⁶⁴Cu and ⁶⁷Cu. In some embodiments, the radionuclide is ⁵⁵Co. In some embodiments the radionuclide is ⁶⁰Cu. In some embodiments the radionuclide is ⁶¹Cu. In some embodiments the radionuclide is ⁶²Cu. In some embodiments the radionuclide is ⁶⁴Cu. In some embodiments the radionuclide is ⁶⁷Cu.

As used herein the term “cancer” broadly encompasses a class of neoplastic diseases characterised with abnormal cell growth with the potential to invade or spread to other parts of the body. In one embodiment, the cancer is one that overexpresses the GRP receptor. These are to be contrasted with benign tumours, which do not spread to other parts of the body and therefore the definition as used herein includes all malignant (cancerous) disease states. The term therefore encompasses the treatment of tumours.

Accordingly, the term “tumour” is used generally to define any malignant cancerous or pre-cancerous cell growth, and may include blood based cancers, but is particularly directed to solid tumours or carcinomas such as prostate cancer, breast cancer, gliomas, gastrointestinal stromal tumours, melanomas, colon, lung, ovarian, skin, pancreas, pharynx, brain, CNS, and renal cancers (as well as other cancers).

The compounds and complexes of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. The compositions are prepared in manners well known in the art.

In using the compounds of the invention they can be administered in any form or mode which makes the compound available for the desired application (imaging or radiotherapy). One skilled in the art of preparing formulations of this type can readily select the proper form and mode of administration depending upon the particular characteristics of the compound selected, the condition to be treated, the stage of the condition to be treated and other relevant circumstances. Reference is made to Remington's Pharmaceutical Sciences, 19^{th} edition, Mack Publishing Co. (1995) for further information.

The invention in other embodiments provides a pharmaceutical pack or kit comprising one or more containers filled with one or more of the ingredients of the pharmaceutical compositions of the invention. In such a pack or kit can be found at least one container having a unit dosage of the agent(s). Conveniently, in the kits, single dosages can be provided in sterile vials so that the clinician can employ the vials directly, where the vials will have the desired amount and concentration of compound and radio nucleotide which may be admixed prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

In an embodiment, the invention provides compositions comprising a compound as described above together with one or more pharmaceutically acceptable excipients.

Pharmaceutical compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

In some embodiments, the formulation is an aqueous formulation and the pharmaceutically acceptable carrier is a saline solution that includes a phosphate buffer. In a preferred embodiment, the pharmaceutically acceptable carrier is sodium phosphate buffer. In a further preferred embodiment, the sodium phosphate buffer is 0.05M sodium phosphate buffer.

In one embodiment, the aqueous formulation comprises a compound of Formula (I), or a salt, isomer, solvate, prodrug or protected form thereof having the structure of Formula (Ia) complexed with a radionuclide:

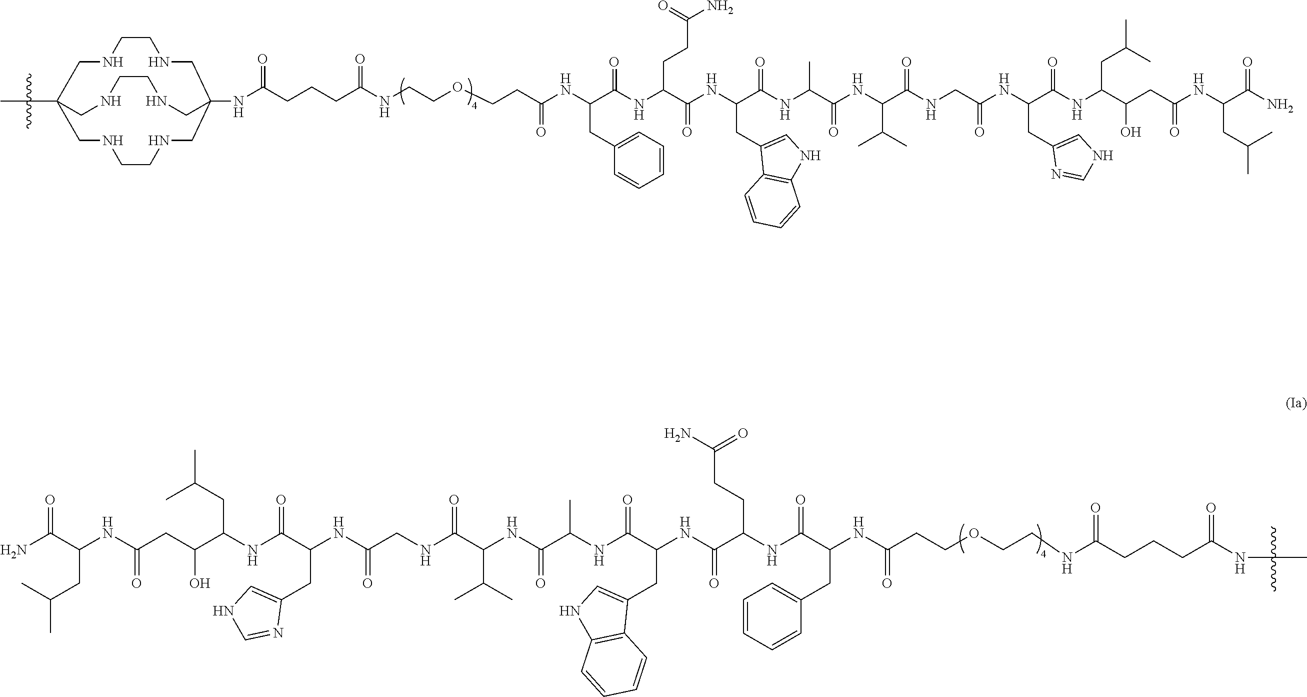

In another embodiment, the aqueous formulation comprises a compound of Formula (I), or a salt, isomer, solvate, prodrug or protected form thereof having the structure of Formula (Ib) complexed with a radionuclide:

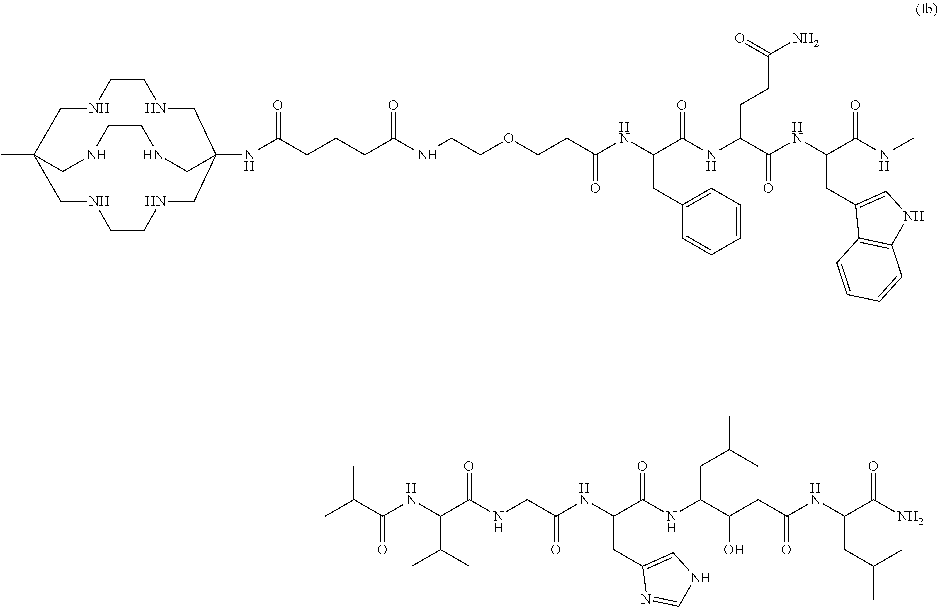

In an embodiment, the aqueous formulation of the present invention comprises one or more stabilizers in a total amount of about 2% to about 10% (w/v). In an embodiment, the aqueous formulation of the present invention comprises one or more stabilizers in a total amount of about 2% (w/v). In another embodiment, the aqueous formulation of the present invention comprises one or more stabilizers in a total amount of about 2.5% (w/v). In another embodiment, the aqueous formulation of the present invention comprises one or more stabilizers in a total amount of about 3% (w/v). In another embodiment, the aqueous formulation of the present invention comprises one or more stabilizers in a total amount of about 3.5% (w/v). In another embodiment, the aqueous formulation of the present invention comprises one or more stabilizers in a total amount of about 4% (w/v). In another embodiment, the aqueous formulation of the present invention comprises one or more stabilizers in a total amount of about 4.5% (w/v). In another embodiment, the aqueous formulation of the present invention comprises one or more stabilizers in a total amount of about 5% (w/v). In another embodiment, the aqueous formulation of the present invention comprises one or more stabilizers in a total amount of about 5.5% (w/v). In another embodiment, the aqueous formulation of the present invention comprises one or more stabilizers in a total amount of about 6% (w/v). In another embodiment, the aqueous formulation of the present invention comprises one or more stabilizers in a total amount of about 6.5% (w/v). In another embodiment, the aqueous formulation of the present invention comprises one or more stabilizers in a total amount of about 7% (w/v). In another embodiment, a formulation of the present invention comprises one or more stabilizers in a total amount of about 7.5% (w/v). In another embodiment, the aqueous formulation of the present invention comprises one or more stabilizers in a total amount of about 8% (w/v). In another embodiment, the aqueous formulation of the present invention comprises one or more stabilizers in a total amount of about 8.5% (w/v). In another embodiment, the aqueous formulation of the present invention comprises one or more stabilizers in a total amount of about 9% (w/v). In another embodiment, the aqueous formulation of the present invention comprises one or more stabilizers in a total amount of about 9.5% (w/v). In another embodiment, the aqueous formulation of the present invention comprises one or more stabilizers in a total amount of about 10% (w/v). In other embodiments, the present invention also contemplates one or more stabilizers present in ranges between the aforementioned amounts.

In an embodiment, the aqueous formulation comprising a compound of Formula (I), or a salt, isomer, solvate, prodrug or protected form thereof complexed to a radionuclide comprises gentisic acid, or a salt thereof, as a stabilizer. Gentisic acid is also known as 2,5-dihydroxybenzoic acid, 5-hydroxysalicylic acid or hydroquinonecarboxylic acid. Salts of gentisic acid may include the sodium salt and the sodium salt hydrate. Any reference to gentisic acid may include a reference to salts thereof, where relevant.

In an embodiment, gentisic acid, or a salt thereof, is present in the formulation in an amount of about 0.01% to about 0.1% (w/v). In an embodiment, gentisic acid, or a salt thereof, is present in the formulation in an amount of about 0.01% (w/v). In an embodiment, gentisic acid, or a salt thereof, is present in the formulation in an amount of about 0.015% (w/v). In an embodiment, gentisic acid, or a salt thereof, is present in the formulation in an amount of about 0.02% (w/v). In another embodiment, gentisic acid, or a salt thereof, is present in the formulation in an amount of about 0.025% (w/v). In another embodiment, gentisic acid, or a salt thereof, is present in the formulation in an amount of about 0.03% (w/v). In another embodiment, gentisic acid, or a salt thereof, is present in the formulation in an amount of about 0.035% (w/v). In another embodiment, gentisic acid, or a salt thereof, is present in the formulation in an amount of about 0.04% (w/v). In another embodiment, gentisic acid, or a salt thereof, is present in the formulation in an amount of about 0.045% (w/v). In another embodiment, gentisic acid, or a salt thereof, is present in the formulation in an amount of about 0.05% (w/v). In another embodiment, gentisic acid, or a salt thereof, is present in the formulation in an amount of about 0.055% (w/v). In another embodiment, gentisic acid, or a salt thereof, is present in the formulation in an amount of about 0.6% (w/v). In another embodiment, gentisic acid, or a salt thereof, is present in the formulation in an amount of about 0.065% (w/v). In another embodiment, gentisic acid, or a salt thereof, is present in the formulation in an amount of about 0.07% (w/v). In another embodiment, gentisic acid, or a salt thereof, is present in the formulation in an amount of about 0.075% (w/v). In another embodiment, gentisic acid, or a salt thereof, is present in the formulation in an amount of about 0.08% (w/v). In another embodiment, gentisic acid, or a salt thereof, is present in the formulation in an amount of about 0.085% (w/v). In another embodiment, gentisic acid, or a salt thereof, is present in the formulation in an amount of about 0.09% (w/v). In another embodiment, gentisic acid, or a salt thereof, is present in the formulation in an amount of about 0.095% (w/v). In another embodiment, gentisic acid, or a salt thereof, is present in the formulation in an amount of about 0.1% (w/v). In other embodiments, the present invention also contemplates gentisic acid, or a salt thereof, in ranges between the aforementioned amounts. In a preferred embodiment, gentisic acid, or a salt thereof, is present in the formulation in an amount of about 0.025% (w/v).

In another embodiment, ascorbic acid, or a salt thereof, is present as a stabilizer in the aqueous formulation. Ascorbic acid is also known as L-ascorbic acid or Vitamin C. Salts of ascorbic acid include sodium ascorbate, calcium ascorbate, potassium ascorbate and sodium ascorbyl phosphate. Derivatives of ascorbic acid are also contemplated. These include fatty acid esters of ascorbic acid, such as the palmitate ester of ascorbic acid, i.e. ascorbyl palmitate.

In an embodiment, ascorbic acid, or a salt thereof is present in an amount of about 3.0% to about 9.0% (w/v). In an embodiment, ascorbic acid, or a salt thereof, is present in the formulation in an amount of about 3.0% (w/v). In another embodiment, ascorbic acid, or a salt thereof, is present in the formulation in an amount of about 3.5% (w/v). In another embodiment, ascorbic acid, or a salt thereof, is present in the formulation in an amount of about 4.0% (w/v). In another embodiment, ascorbic acid, or a salt thereof, is present in the formulation in an amount of about 4.5% (w/v). In another embodiment, ascorbic acid, or a salt thereof, is present in the formulation in an amount of about 5.0% (w/v). In another embodiment, ascorbic acid, or a salt thereof, is present in the formulation in an amount of about 5.5% (w/v). In another embodiment, ascorbic acid, or a salt thereof, is present in the formulation in an amount of about 6.0% (w/v). In another embodiment, ascorbic acid, or a salt thereof, is present in the formulation in an amount of about 6.5% (w/v). In another embodiment, ascorbic acid, or a salt thereof, is present in the formulation in an amount of about 7.0% (w/v). In another embodiment, ascorbic acid, or a salt thereof, is present in the formulation in an amount of about 7.5% (w/v). In another embodiment, ascorbic acid, or a salt thereof, is present in the formulation in an amount of about 8.0% (w/v). In another embodiment, ascorbic acid, or a salt thereof, is present in the formulation in an amount of about 8.5% (w/v). In another embodiment, ascorbic acid, or a salt thereof, is present in the formulation in an amount of about 9.0% (w/v). In other embodiments, the present invention also contemplates ascorbic acid, or a salt thereof, in ranges between the aforementioned amounts. In a preferred embodiment, ascorbic acid, or a salt thereof, is present in the formulation in an amount of about 6.25% (w/v).

L-methionine, or a salt thereof, may also be utilised as a stabiliser. The term L-methionine as used herein refers to the amino acid bearing an S-methyl thioether side chain. The addition of L-methionine to a formulation of the present invention may further enhance the stability of the formulation by preventing or minimising radiolysis of a radiolabelled complex of Formula (I), thereby increasing the radiochemical purity of the formulation.

The aqueous formulations of the present invention may also comprise ethanol as a component. The ethanol used in the formulation may be anhydrous ethanol. Alternatively, the ethanol used in the aqueous formulation may not have been subject to drying processes and may be hydrated. In certain embodiments, the ethanol is aqueous ethanol. The ethanol is preferably pharmaceutical grade ethanol. The ethanol present in the formulation may further assist in preventing radiolysis of the radiolabelled complex of Formula (I).

In an embodiment, ethanol is present in the aqueous formulation in an amount of about 1% to about 7% (v/v). In an embodiment, ethanol is present in the formulation in an amount of about 1% (v/v). In another embodiment, ethanol is present in the formulation in an amount of about 1.5% (v/v). In another embodiment, ethanol is present in the formulation in an amount of about 2% (v/v). In another embodiment, ethanol is present in the formulation in an amount of about 2.5% (v/v). In another embodiment, ethanol is present in the formulation in an amount of about 3% (v/v). In another embodiment, ethanol is present in the formulation in an amount of about 3.5% (v/v). In another embodiment, ethanol is present in the formulation in an amount of about 4% (v/v). In another embodiment, ethanol is present in the formulation in an amount of about 4.5% (v/v). In another embodiment, ethanol is present in the formulation in an amount of about 5% (v/v). In another embodiment, ethanol is present in the formulation in an amount of about 5.5% (v/v). In another embodiment, ethanol is present in the formulation in an amount of about 6% (v/v). In another embodiment, ethanol is present in the formulation in an amount of about 6.5% (v/v). In another embodiment, ethanol is present in the formulation in an amount of about 7% (v/v). In a preferred embodiment, ethanol is present in the formulation in an amount of about 4% (v/v). In other embodiments, the present invention also contemplates ethanol in ranges between the aforementioned amounts.

In one embodiment, the invention provides an aqueous formulation comprising a compound of Formula (I), or a salt, isomer, solvate, prodrug or protected form thereof, complexed with a radionuclide:

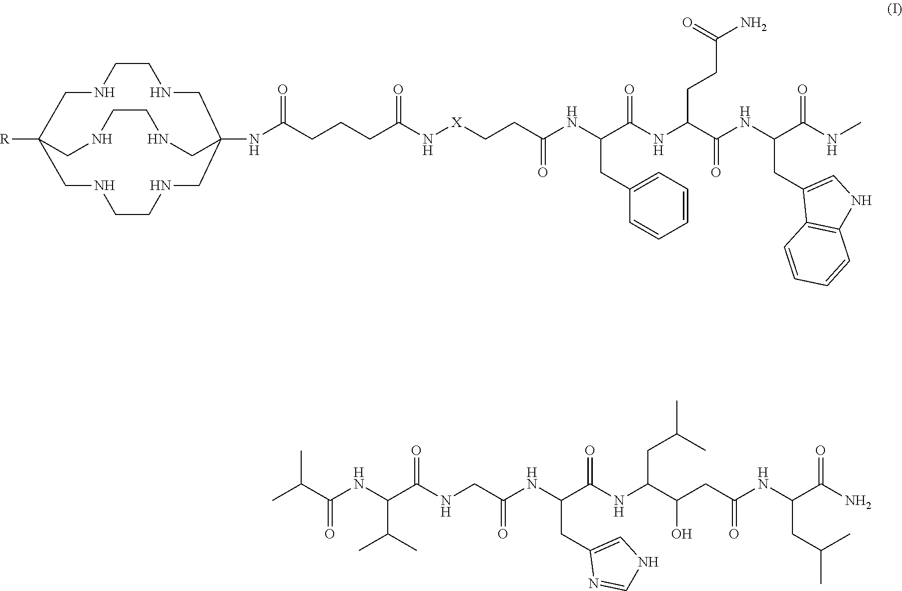

wherein:
X is

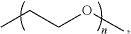

where n is an integer from 1 to 10; and
R is a group selected from the group consisting of H, OH, halogen, cyano, NO₂, NH₂, optionally substituted C₁-C₁₂ alkyl, optionally substituted amino, optionally substituted amide, and an optionally substituted aryl;
the formulation comprising a buffer and one or more excipients.

In a further embodiment, the aqueous formulation comprises sodium phosphate buffer, gentisic acid, or a salt thereof, ethanol and ascorbic acid, or a salt thereof.

In yet another embodiment, the aqueous formulation comprises sodium phosphate buffer, about 0.01% to about 0.1% (w/v) gentisic acid, or a salt thereof, about 1% to about 7% (v/v) ethanol, and about 3.0% to about 9.0% (w/v) ascorbic acid, or a salt thereof.

In one embodiment, the radionuclide is selected from the group consisting of ⁶⁰Cu, ⁶¹Cu, ⁶²Cu, and ⁶⁴Cu.

In a preferred embodiment, the radionuclide is ⁶⁴Cu. In another preferred embodiment, for the compound of Formula (I) R is methyl, X is

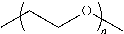

and n is 4.

Accordingly, in one embodiment, the present invention provides an aqueous formulation comprising a compound of Formula (Ib), or a salt, isomer, solvate, prodrug or protected form thereof, complexed with ⁶⁴Cu:

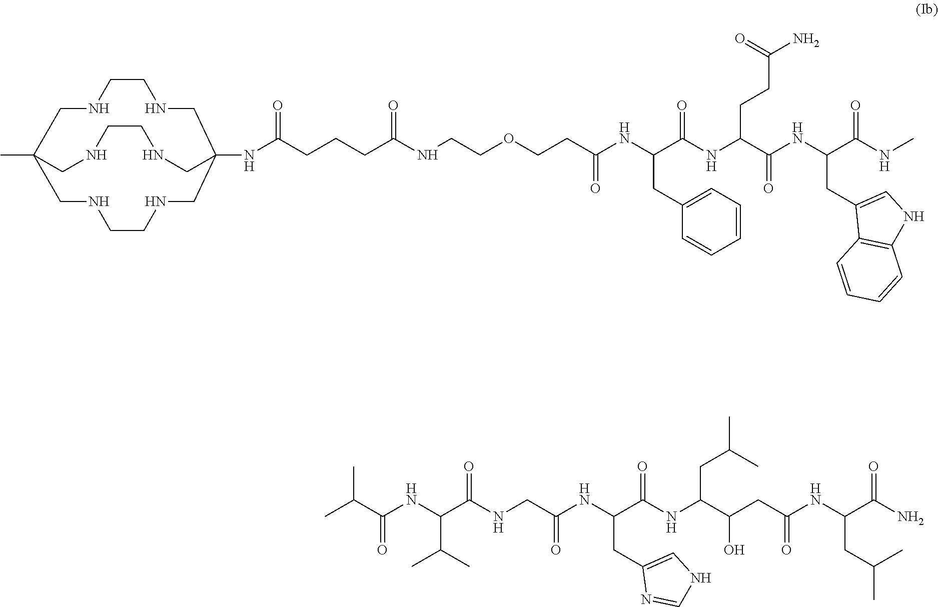

wherein:
X is

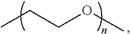

where n is 4;
the formulation comprising sodium phosphate buffer, about 0.01% to about 0.1% (w/v) gentisic acid, or a salt thereof, 1% to about 7% (v/v) ethanol, and about 3.0% to about 9.0% (w/v) ascorbic acid, or a salt thereof.

The formulations of the present invention have a pH of about 4 to about 8. A person skilled in the art would understand that the pH of the formulation is an inherent characteristic of the formulation, attributed to the combination of the compound of Formula (I) or a complex thereof, and the remaining excipients of the formulation. The present inventors have found that this pH range provides for optimal radiolabelling efficiency, and also stability of the radiolabelled complex both in the formulation and when administered in vivo.

In an embodiment, the pH of the formulation is from about 4 to about 8. In an embodiment, the pH of the formulation is about 4. In another embodiment, the pH of the formulation is about 4.5. In another embodiment, the pH of the formulation is about 5.0. In an embodiment, the pH of the formulation is about 5.5. In another embodiment, the pH of the formulation is about 5.6. In another embodiment, the pH of the formulation is about 5.7. In another embodiment, the pH of the formulation is about 5.8. In another embodiment, the pH of the formulation is about 5.9. In another embodiment, the pH of the formulation is about 6.0. In another embodiment, the pH of the formulation is about 6.1. In another embodiment, the pH of the formulation is about 6.2. In another embodiment, the pH of the formulation is about 6.3. In another embodiment, the pH of the formulation is about 6.4. In another embodiment, the pH of the formulation is about 6.5. In another embodiment, the pH of the formulation is about 7.0. In another embodiment, the pH of the formulation is about 7.5. In another embodiment, the pH of the formulation is about 8.0. In a preferred embodiment, the pH of the formulation is about 6.0.

Aqueous formulations of the invention may be prepared, for example, by adding the nucleotide to the compound of Formula (I), or the salt, isomer, solvate, prodrug or protected form thereof in a solution of sodium phosphate buffer and gentisic acid, or a salt thereof, and incubating the solution for an appropriate time. The solution may then be filtered and quenched by addition of an aqueous ethanol solution containing ascorbic acid, or a salt thereof, then filtered into a sterile vial prior to injection into a subject in need thereof.

In one embodiment, the aqueous formulation is prepared adding ⁶⁴Cu to the compound of Formula (I), or the salt, isomer, solvate, prodrug or protected form thereof in sodium phosphate buffer and about 0.01% to about 0.1% (w/v) gentisic acid, or a salt thereof, incubating the solution for an appropriate time, filtering the solution, then quenching the reaction by addition of an aqueous solution comprising about 1% to about 7% (v/v) ethanol, and about 3.0% to about 9.0% (w/v) ascorbic acid, or a salt thereof. The formulation is then filtered into a sterile vial prior to injection into a subject in need thereof.

As mentioned above, a preferred embodiment of the present invention is an aqueous formulation comprising a complex of a compound of Formula (I), or a salt, isomer, solvate, prodrug or protected form thereof and the radionuclide ⁶⁴Cu. A formulation of a complex of ⁶⁴Cu and a compound of Formula (I) may have a radiochemical purity of at least about 90% for a time of at least 48 hours. This means that at least about 90% of the ⁶⁴Cu radioisotope present in the formulation is complexed with the compound of Formula (I), or a salt thereof, for at least 48 hours after preparation of the formulation. Where the ⁶⁴Cu radioisotope present in the formulation is not complexed with the compound of Formula (I), or a salt thereof, the ⁶⁴Cu radioisotope may be present as a free ⁶⁴Cu ion, or as part of a radiolysis product.

In an embodiment, the radiochemical purity of a formulation of the present invention comprising a complex of ⁶⁴Cu and a compound of Formula (I), or a salt, isomer, solvate, prodrug or protected form thereof is about 90% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 91% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 92% at a time of about 48 hours after preparation of the formulation.

In another embodiment, the radiochemical purity of a formulation of the present invention comprising a complex of ⁶⁴Cu and a compound of Formula (I), or a salt, isomer, solvate, prodrug or protected form thereof is about 93% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 94% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 95% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 97% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 98% at a time of about 48 hours after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 99% at a time of about 48 hours after preparation of the formulation.

In an embodiment, the radiochemical purity of a formulation of the present invention comprising a complex of ⁶⁴Cu and a compound of Formula (I), or a salt, isomer, solvate, prodrug or protected form thereof is about 96% immediately after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 1 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 3 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 6 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 9 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 12 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 15 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 18 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 21 h after preparation of the formulation. In another embodiment, the radiochemical purity of a formulation of the present invention comprising a complex of ⁶⁴Cu and a compound of Formula (I), or a salt, isomer, solvate, prodrug or protected form thereof is about 96% after about 24 h after preparation of the formulation.

### Preparation of an Aqueous Formulation of the Present Invention

The compound of Formula (I), or the salt, isomer, solvate, prodrug or protected form thereof, complexed with a radionuclide may be provided by mixing a compound of Formula (I), or a salt, isomer, solvate, prodrug or protected form thereof, with a solution of a radionuclide in the presence of a buffer and one or more stabilizing agents. The solution may then be filtered and the reaction subsequently quenched to provide the formulation comprising a compound of Formula (I), or a salt, isomer, solvate, prodrug or protected form thereof, complexed with a radionuclide. In one embodiment, the stabilizing agent is gentisic acid, or a salt thereof. In one embodiment, the reaction between the compound of Formula (I) and the radionuclide is quenched with an aqueous ethanol solution comprising ascorbic acid, or a salt thereof.

Accordingly, the present invention provides a process for preparing an aqueous formulation comprising a compound of Formula (I), or a salt, isomer, solvate, prodrug or protected form thereof complexed with a radionuclide, the method comprising the steps of:
i) dissolving a compound of Formula (I), or a salt, isomer, solvate, prodrug or protected form thereof, in a buffer solution comprising gentisic acid, or a salt thereof;
ii) adding a solution of a radionuclide to the solution of step i);
iii) filtering the solution obtained from step ii); and
iv) quenching the reaction by addition of aqueous ethanol and ascorbic acid;
   to recover an aqueous formulation comprising a compound of Formula (I), or a salt, isomer, solvate, prodrug or protected form thereof, complexed with a radionuclide.

The buffer may be a solution of ammonium acetate. Alternatively, the buffer solution may be a solution of sodium acetate. In a preferred embodiment, the buffer solution is a sodium phosphate buffer.

The buffer solution comprises gentisic acid, or a salt thereof, as a component. As previously described, salts of gentisic acid may include the sodium salt or the sodium salt hydrate. Other salts of gentisic acid are also contemplated. The buffer solution may comprise sodium gentisate at a concentration of between about 0.01 to about 0.1% (w/v). In an embodiment, the buffer solution comprises sodium gentisate at a concentration of about 0.01% (w/v). In another embodiment, the buffer solution comprises sodium gentisate at a concentration of about 0.015% (w/v). In another embodiment, the buffer solution comprises sodium gentisate at a concentration of about 0.02% (w/v). In another embodiment, the buffer solution comprises sodium gentisate at a concentration of about 0.025% (w/v). In another embodiment, the buffer solution comprises sodium gentisate at a concentration of about 0.03% (w/v). In another embodiment, the buffer solution comprises sodium gentisate at a concentration of about 0.035% (w/v). In another embodiment, the buffer solution comprises sodium gentisate at a concentration of about 0.04% (w/v). In another embodiment, the buffer solution comprises sodium gentisate at a concentration of about 0.045% (w/v). In another embodiment, the buffer solution comprises sodium gentisate at a concentration of about 0.05% (w/v). In another embodiment, the buffer solution comprises sodium gentisate at a concentration of about 0.055% (w/v). In another embodiment, the buffer solution comprises sodium gentisate at a concentration of about 0.06% (w/v). In another embodiment, the buffer solution comprises sodium gentisate at a concentration of about 0.065% (w/v). In another embodiment, the buffer solution comprises sodium gentisate at a concentration of about 0.07% (w/v). In another embodiment, the buffer solution comprises sodium gentisate at a concentration of about 0.075% (w/v). In another embodiment, the buffer solution comprises sodium gentisate at a concentration of about 0.08% (w/v). In another embodiment, the buffer solution comprises sodium gentisate at a concentration of about 0.085% (w/v). In another embodiment, the buffer solution comprises sodium gentisate at a concentration of about 0.095% (w/v). In another embodiment, the buffer solution comprises sodium gentisate at a concentration of about 0.1% (w/v). In a preferred embodiment, the buffering solution comprises sodium gentisate at a concentration of about 0.035% to 0.04% (w/v).

The reaction between the compound of Formula (I), or the salt, isomer, solvate, prodrug or protected form thereof, and the radionuclide is quenched with an aqueous ethanol solution. As previously described, the ethanol may be anhydrous or may be previously subjected to drying procedures known in the art. The solution may comprise ethanol at a concentration of between about 1 to about 7% (v/v). In an embodiment, the solution comprises ethanol at a concentration of about 1% (v/v). In another embodiment, the solution comprises ethanol at a concentration of about 1.5% (v/v). In another embodiment, the solution comprises ethanol at a concentration of about 2% (v/v). In another embodiment, the solution comprises ethanol at a concentration of about 2.5% (v/v). In another embodiment, the solution comprises ethanol at a concentration of about 3% (v/v). In another embodiment, the solution comprises ethanol at a concentration of about 3.5% (v/v). In another embodiment, the solution comprises ethanol at a concentration of about 4% (v/v). In another embodiment, the solution comprises ethanol at a concentration of about 4.5% (v/v). In another embodiment, the solution comprises ethanol at a concentration of about 5% (v/v). In another embodiment, the solution comprises ethanol at a concentration of about 5.5% (v/v). In another embodiment, the solution comprises ethanol at a concentration of about 6% (v/v). In another embodiment, the buffering solution comprises ethanol at a concentration of about 6.5% (v/v). In another embodiment, the buffering solution comprises ethanol at a concentration of about 7% (v/v). In a preferred embodiment, the buffering solution comprises ethanol at a concentration of about 4% (v/v).

As mentioned above, the aqueous ethanol solution comprises ascorbic acid, or a salt thereof. Ascorbic acid is also known as L-ascorbic acid or Vitamin C. Salts of ascorbic acid include sodium ascorbate, calcium ascorbate, potassium ascorbate and sodium ascorbyl phosphate. Derivatives of ascorbic acid are also contemplated. These include fatty acid esters of ascorbic acid, such as the palmitate ester of ascorbic acid, i.e. ascorbyl palmitate. In an embodiment, ascorbic acid, or a salt thereof is present in an amount of about 3.0% to about 9.0% (w/v). In an embodiment, ascorbic acid, or a salt thereof, is present in the solution in an amount of about 3.0% (w/v). In another embodiment, ascorbic acid, or a salt thereof, is present in the solution in an amount of about 3.5% (w/v). In another embodiment, ascorbic acid, or a salt thereof, is present in the solution in an amount of about 4.0% (w/v). In another embodiment, ascorbic acid, or a salt thereof, is present in the solution in an amount of about 4.5% (w/v). In another embodiment, ascorbic acid, or a salt thereof, is present in the solution in an amount of about 5.0% (w/v). In another embodiment, ascorbic acid, or a salt thereof, is present in the solution in an amount of about 5.5% (w/v). In another embodiment, ascorbic acid, or a salt thereof, is present in the solution in an amount of about 6.0% (w/v). In another embodiment, ascorbic acid, or a salt thereof, is present in the solution in an amount of about 6.5% (w/v). In another embodiment, ascorbic acid, or a salt thereof, is present in the solution in an amount of about 7.0% (w/v). In another embodiment, ascorbic acid, or a salt thereof, is present in the solution in an amount of about 7.5% (w/v). In another embodiment, ascorbic acid, or a salt thereof, is present in the solution in an amount of about 8.0% (w/v). In another embodiment, ascorbic acid, or a salt thereof, is present in the solution in an amount of about 8.5% (w/v). In another embodiment, ascorbic acid, or a salt thereof, is present in the solution in an amount of about 9.0% (w/v). In a preferred embodiment, ascorbic acid, or a salt thereof, is present in the solution in an amount of about 6.25% (w/v).

According to an embodiment of the present invention, a compound of Formula (I), or a salt, isomer, solvate, prodrug or protected form thereof, is mixed in a sodium phosphate buffer solution comprising gentisic acid, or a salt thereof. The compound of Formula (I) or a salt thereof, may be obtained as a solid. In an embodiment, the compound of Formula (I) or a salt, isomer, solvate, prodrug or protected form thereof, is obtained as a lyophilised powder. In an embodiment, the compound of Formula (I) or a salt thereof, obtained as a lyophilised powder is mixed with a sodium phosphate buffer solution comprising gentisic acid or a salt thereof. In an embodiment, about 1 μg to about 180 μg of the compound of Formula (I) or a salt, isomer, solvate, prodrug or protected form thereof, as a lyophilised powder is mixed with a sodium phosphate buffer solution comprising gentisic acid or a salt thereof.

A solution of the radionuclide is added to the mixture of a compound of Formula (I), or a salt, isomer, solvate, prodrug or protected form thereof, and the sodium phosphate buffer solution comprising gentisic acid or a salt thereof, and is allowed to stand for a time.

In an embodiment, the solution of a Cu ion is a solution of a Cu salt. In another embodiment, the solution of a Cu ion is a solution of a chloride salt containing copper. In another embodiment, the solution of a Cu ion is a solution of a copper(II) chloride salt. In another embodiment, the solution of a Cu ion is a solution of a copper salt containing a ⁶⁰Cu radioisotope. In another embodiment, the solution of a Cu ion is a solution of a chloride salt containing a ⁶¹Cu radioisotope. In another embodiment, the solution of a Cu ion is a solution of a chloride salt containing a ⁶⁴Cu radioisotope. In another embodiment, the solution of a Cu ion is a solution of a radioactive copper(II) chloride salt. In another embodiment, the solution of a Cu ion is a solution of a copper(II) chloride salt, wherein the copper is the ⁶¹Cu isotope. In another embodiment, the solution of a Cu ion is a solution of a copper(II) chloride salt, wherein the copper is the ⁶⁴Cu isotope. In another embodiment, the solution of Cu ion is a solution of [⁶¹Cu]CuCl₂. In another embodiment, the solution of a Cu ion is a solution of [⁶⁴Cu]CuCl₂.

The solution of a Cu ion is provided as an aqueous solution. The Cu ion may be provided in an aqueous solution of hydrochloric acid. In an embodiment, the Cu ion is provided in a solution of between about 0.01 to about 0.1 mol/L hydrochloric acid. In an embodiment, the Cu ion is provided in a solution of about 0.01 mol/L hydrochloric acid. In another embodiment, the Cu ion is provided in a solution of about 0.02 mol/L hydrochloric acid. In another embodiment, the Cu ion is provided in a solution of about 0.05 mol/L hydrochloric acid. In another embodiment, the Cu ion is provided in a solution of about 0.075 mol/L hydrochloric acid. In another embodiment, the Cu ion is provided in a solution of about 0.1 mol/L hydrochloric acid.

In a preferred embodiment, the Cu ion is provided as [⁶⁴Cu]CuCl₂ in a solution of about 0.05 mol/L hydrochloric acid. The solution of a ⁶⁴Cu-radioisotope is provided as an aqueous solution with a radioactivity of between about 50 to about 10,000 MBq. In an embodiment, the radioactivity of the ⁶⁴Cu-radioisotope solution is about 50 MBq. In another embodiment, the radioactivity of the ⁶⁴Cu-radioisotope solution is about 100 MBq. In another embodiment, the radioactivity of the ⁶⁴Cu-radioisotope solution is about 200 MBq. In another embodiment, the radioactivity of the ⁶⁴Cu-radioisotope solution is about 300 MBq. In another embodiment, the radioactivity of the ⁶⁴Cu-radioisotope solution is about 400 MBq. In another embodiment, the radioactivity of the ⁶⁴Cu-radioisotope solution is about 500 MBq. In another embodiment, the radioactivity of the ⁶⁴Cu-radioisotope solution is about 600 MBq. In another embodiment, the radioactivity of the ⁶⁴Cu-radioisotope solution is about 700 MBq. In another embodiment, the radioactivity of the ⁶⁴Cu-radioisotope solution is about 800 MBq. In another embodiment, the radioactivity of the ⁶⁴Cu-radioisotope solution is about 900 MBq. In another embodiment, the radioactivity of the ⁶⁴Cu-radioisotope solution is about 1,000 MBq. In another embodiment, the radioactivity of the ⁶⁴Cu-radioisotope is about 1,500 MBq. In another embodiment, the radioactivity of the ⁶⁴Cu-radioisotope is about 2,000 MBq. In another embodiment, the radioactivity of the ⁶⁴Cu-radioisotope is about 2,500 MBq. In another embodiment, the radioactivity of the ⁶⁴Cu-radioisotope is about 3,000 MBq. In another embodiment, the radioactivity of the ⁶⁴Cu-radioisotope is about 3,500 MBq. In another embodiment, the radioactivity of the ⁶⁴Cu-radioisotope is about 4,000 MBq. In another embodiment, the radioactivity of the ⁶⁴Cu-radioisotope is about 4,500 MBq. In another embodiment, the radioactivity of the ⁶⁴Cu-radioisotope is about 5,000 MBq. In another embodiment, the radioactivity of the ⁶⁴Cu-radioisotope is about 5,500 MBq. In another embodiment, the radioactivity of the ⁶⁴Cu-radioisotope is about 6,000 MBq. In another embodiment, the radioactivity of the ⁶⁴Cu-radioisotope is about 6,500 MBq. In another embodiment, the radioactivity of the ⁶⁴Cu-radioisotope is about 7,000 MBq. In another embodiment, the radioactivity of the ⁶⁴Cu-radioisotope is about 7,500 MBq. In another embodiment, the radioactivity of the ⁶⁴Cu-radioisotope is about 8,000 MBq. In another embodiment, the radioactivity of the ⁶⁴Cu-radioisotope is about 8,500 MBq. In another embodiment, the radioactivity of the ⁶⁴Cu-radioisotope is about 9,000 MBq. In another embodiment, the radioactivity of the ⁶⁴Cu-radioisotope is about 9,500 MBq. In another embodiment, the radioactivity of the ⁶⁴Cu-radioisotope is about 10,000 MBq.

A mixture of a radionuclide, a compound of Formula (I), or a salt, isomer, solvate, prodrug or protected form thereof, and the sodium phosphate buffer solution comprising gentisic acid, or a salt thereof, may be allowed to stand at room temperature. The mixture may be allowed to stand with stirring, alternatively, the mixture is allowed to stand without stirring. The mixture may be allowed to stand for a time between about 5 to about 60 minutes. In an embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 5 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 10 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 15 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 20 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 25 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 30 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 35 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 40 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 45 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 50 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 55 minutes. In another embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 60 minutes. In a preferred embodiment, the mixture of a radionuclide, a compound of Formula (I), or a salt, isomer, solvate, prodrug or protected form thereof, and the sodium phosphate buffer solution comprising gentisic acid, or a salt thereof is allowed to stand without stirring for about 25 minutes.

According to another embodiment of the present invention, the mixture of a radionuclide, a compound of Formula (I), or a salt, isomer, solvate, prodrug or protected form thereof, and the sodium phosphate buffer solution comprising gentisic acid, or a salt thereof, is filtered. The mixture may be filtered through a solid phase extraction process. The mixture may be filtered through a solid phase extraction process, where the stationary phase of the solid phase extraction cartridge retains the compound of Formula (I), or a salt, isomer, solvate, prodrug or protected form thereof, complexed with a Cu ion, any compound of Formula (I), or a salt, isomer, solvate, prodrug or protected form thereof, that is not complexed and some gentisic acid in the form of a salt that is present, such as sodium gentisate. As used herein, the term “stationary phase” refers to a resin-like material that is held within the solid phase extraction cartridge and allows for the separation of compounds based on their polarity.

The solid phase extraction process as described herein may use a reverse-phase stationary phase. As used herein, the term “reverse-phase” in relation to a stationary phase refers to a stationary phase that is hydrophobic in nature, such that the stationary phase has an affinity for hydrophobic or uncharged molecules. Examples of a reverse-phase stationary phase may include Phenomenex Strata-X 33u Polymeric Reversed Phase, Waters tC18 or Waters C18. Other similar stationary phases may be used. As the solid phase extraction process uses a reverse-phase stationary phase, any free radionuclide ions and the remaining gentisic acid or its salt is not retained by the stationary phase and these components are discarded.

In an embodiment, the mixture of a radionuclide, a compound of Formula (I) or a salt, isomer, solvate, prodrug or protected form thereof and the sodium phosphate buffer solution comprising gentisic acid, or a salt thereof is filtered through a solid phase extraction cartridge. In an embodiment, the mixture of a radionuclide, a compound of Formula (I) or a salt, isomer, solvate, prodrug or protected form thereof and the sodium phosphate buffer solution comprising gentisic acid, or a salt thereof, is filtered through a solid phase extraction cartridge with a reverse-phase stationary phase. In an embodiment, the compound of Formula (I) or a salt, isomer, solvate, prodrug or protected form thereof complexed with a radionuclide is retained by a solid phase extraction cartridge with a reverse-phase stationary phase. In a preferred embodiment, the mixture of a radionuclide, a compound of Formula (I) or a salt, isomer, solvate, prodrug or protected form thereof and the sodium phosphate buffer solution comprising gentisic acid, or a salt thereof is filtered through a solid phase extraction cartridge with reverse-phase stationary phase. In another preferred embodiment, the compound of Formula (I) or a salt, isomer, solvate, prodrug or protected form thereof complexed with a radionuclide is retained by a solid phase extraction cartridge with a reverse-phase stationary phase.

The compound of Formula (I) or the salt, isomer, solvate, prodrug or protected form thereof complexed with a radionuclide is eluted from the solid phase extraction cartridge containing the stationary phase by washing with a solvent. As the solid phase extraction cartridge contains a reverse-phase stationary phase, eluting the compound of Formula (I) or the salt, isomer, solvate, prodrug or protected form thereof complexed with a radionuclide requires washing of the stationary phase with ethanol, saline and/or another solvent. In an embodiment, the solid phase extraction cartridge is washed with ethanol to elute the compound of Formula (I) or the salt, isomer, solvate, prodrug or protected form thereof complexed with a radionuclide. In another embodiment, the solid phase extraction cartridge is washed with saline to elute the compound of Formula (I) or the salt, isomer, solvate, prodrug or protected form thereof complexed with a radionuclide. In another embodiment, the solid phase extraction cartridge is washed with ethanol and saline to elute the compound of Formula (I) or the salt, isomer, solvate, prodrug or protected form thereof complexed with a radionuclide. In a preferred embodiment, the solid phase extraction cartridge is washed with ethanol and comprising ascorbic acid to elute the compound of Formula (I) or the salt, isomer, solvate, prodrug or protected form thereof complexed with a radionuclide. In a preferred embodiment, the solid phase extraction cartridge is washed with ethanol comprising ascorbic acid to provide the formulation of the present invention.

A person skilled in the art would understand that the excipients of the formulation include the solvent that is used to elute the compound of Formula (I) or the salt, isomer, solvate, prodrug or protected form thereof complexed with a radionuclide from the stationary phase, and that the amount of each solvent used is related to the amount of each excipient in the formulations of the present invention.

A person skilled in the art would understand that the present disclosure provides a manual process for producing a formulation according to the present invention. A person skilled in the art would understand that the steps described herein may be automated, by using a suitable automated radiosynthesis module, in order to obtain a formulation according to the present invention.

The present inventors have found that the formulations disclosed herein have greater stability and show reduced radiolysis in light of the higher starting radioactivity. This enhanced stability may be attributed to the increased radiochemical purity of the formulation at a given radioactivity. The stability of the formulations of the present invention may be observed for a time of up to 90 hours post-manufacture for a formulation. Where the formulations of the present invention are used for the purposes of treatment or therapy, the greater stability may mean that doses for multiple patients at multiple remote locations can be prepared at the same time at a single facility. This may mean that resources for manufacture are required at a single facility, rather than at multiple facilities, and greater efficiency in production of the formulations may be achieved. Where the formulations of the present invention are used for imaging purposes, further advantages may be provided since the clinical imaging sites can receive a dosage form that is ready to inject. This may be particularly advantageous for clinical sites where dedicated radiopharmaceutical production facilities do not exist.

The formulations of the present invention comprise a ligand-radioisotope complex, where the ligand is a compound of Formula (I), or a salt, isomer, solvate, prodrug or protected form thereof. The compound of Formula (I), or a salt, isomer, solvate, prodrug or protected form thereof, and the radioisotope may be supplied in separate containers. Alternatively, the compound of Formula (I), or a salt, isomer, solvate, prodrug or protected form thereof, and the radioisotope may be supplied together as a ligand-radioisotope complex.

The container consisting of the compound of Formula (I) or a salt, isomer, solvate, prodrug or protected form thereof, may provide the compound of Formula (I) as a lyophilised powder. The container may be provided at a temperature of between −20° C. and 20° C.

The formulations may be provided as a kit comprising a container of the radioisotope and a separate container with the ligand and instructions for making the aqueous formulation of the present invention. In an embodiment, the kit of the present invention comprises a container providing a solution of a radioisotope and a separate container providing a compound of Formula (I) or a salt, isomer, solvate, prodrug or protected form thereof. The container providing the radioisotope may contain the radioisotope as a salt.

In an embodiment, a kit of the present invention comprises a container with a solution of radioisotope. In another embodiment, a kit of the present invention comprises a container with a solution of a salt of the radioisotope. In another embodiment, a kit of the present invention comprises a container with a solution of a chloride salt containing the radioisotope. In another embodiment, a kit of the present invention comprises a container with a solution of a radioactive copper(II) chloride salt. In another embodiment, a kit of the present invention comprises a container with a solution of a copper(II) chloride salt, wherein the copper ion is the ⁶⁴Cu isotope. In another embodiment, a kit of the present invention comprises a container with a solution of [⁶⁴Cu]CuCl₂.

The solution of the radioisotope is typically provided as an aqueous solution. In an embodiment, a kit of the present invention provides a radioisotope in the form of an aqueous solution. In a further embodiment, a kit of the present invention provides a radioisotope in the form of an acidic aqueous solution. In another embodiment, a kit of the present invention provides a radioisotope as a solution in hydrochloric acid. The radioisotope may be provided as a solution in hydrochloric acid at a concentration of between about 0.01 and about 0.1 mol/L.

In an embodiment, a kit of the present invention comprises a container with a solution of the radioisotope in hydrochloric acid. In another embodiment, a kit of the present invention comprises a container with a solution of the radioisotope in hydrochloric acid, wherein the hydrochloric acid is at a concentration of about 0.02 mol/L. In another embodiment, a kit of the present invention comprises a container with a solution of the radioisotope in hydrochloric acid, wherein the hydrochloric acid is at a concentration of about 0.05 mol/L. In another embodiment, a kit of the present invention comprises a container with a solution of the radioisotope in hydrochloric acid, wherein the hydrochloric acid is at a concentration of about 0.1 mol/L.

The kit may further comprise a container consisting of sodium phosphate buffer, ethanol, gentisic acid, or a salt thereof and ascorbic acid, or a salt thereof. The kit may comprise a container consisting of sodium phosphate buffer and gentisic acid in an aqueous solution and a second container consisting of a solution of aqueous ethanol and ascorbic acid, or a salt thereof or alternatively, the container may consist only of ethanol, ascorbic acid, or a salt thereof and gentisic acid, or a salt thereof. In an embodiment, the kit comprises a container comprising sodium phosphate buffer and gentisic acid, or a salt thereof and a second container comprising aqueous ethanol and ascorbic acid, or a salt thereof.

In a further embodiment, the invention provides a method of treating a cancer in a subject, the method comprising administering to a subject in need thereof the aqueous formulation according to the invention.

The present invention also provides processes for the synthesis or preparation of compounds of the invention.

The present inventors have found that following established procedures for the preparation of compounds of the present invention by various coupling steps results in the unwanted modification of the terminal amino acid of the bombesin-like peptide. It has been observed that as a result of the coupling reaction depicted in the scheme below (i.e. under standard peptide coupling conditions), the terminal amide of the bombesin-like peptide is converted to the corresponding carboxylic acid.

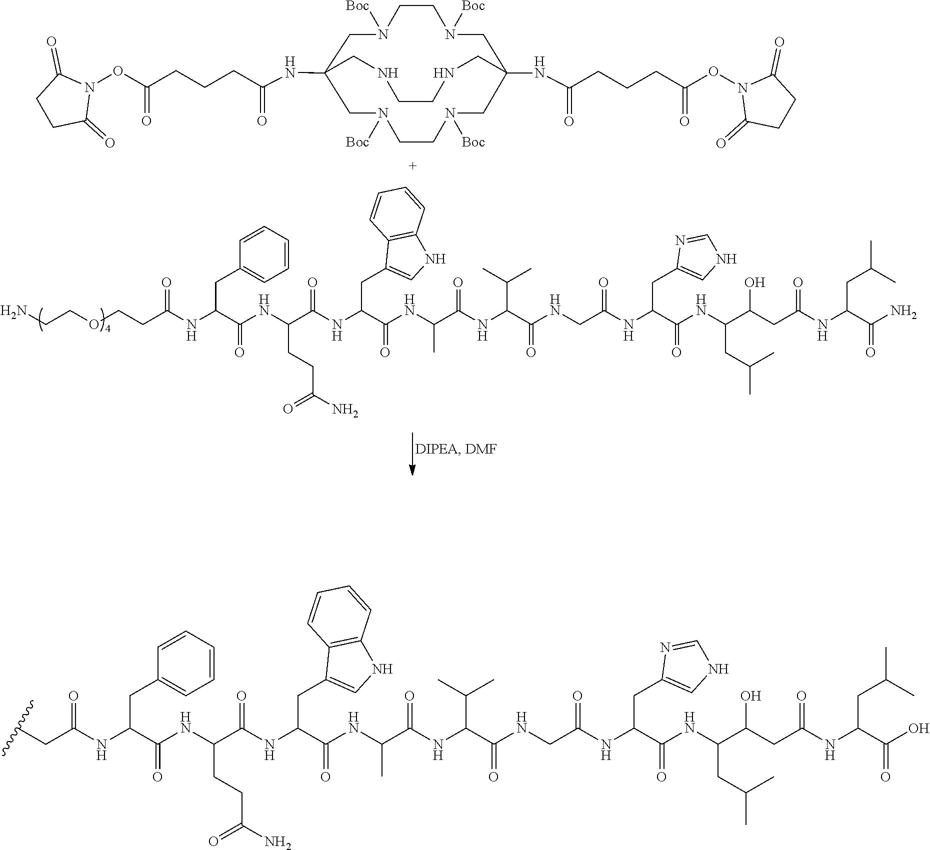

Since the terminal amide group is vital for binding of the bombesin-like peptide (and subsequently the compound as a whole) to the GRP receptor, the modification and deactivation of the bombesin-like peptide during synthesis of compounds of the present invention is undesirable.

The present inventors have found that the coupling reaction depicted in Scheme 1 below under microwave conditions results in the compound of Formula (I) being produced without modification of the terminal amide group. This means that compounds of Formula (I) where the bombesin-like peptide is maintained in its intended form, i.e. without conversion of the terminal amide group to the corresponding carboxylic acid, may now be accessed. The present inventors have also found that where the symmetrical compounds of Formula (I) containing two bombesin-like peptides are produced under suitable microwave conditions, the compounds can be synthesized in a “one-pot” manner, i.e. where both coupling reactions take place at the same time in a single reaction vessel.

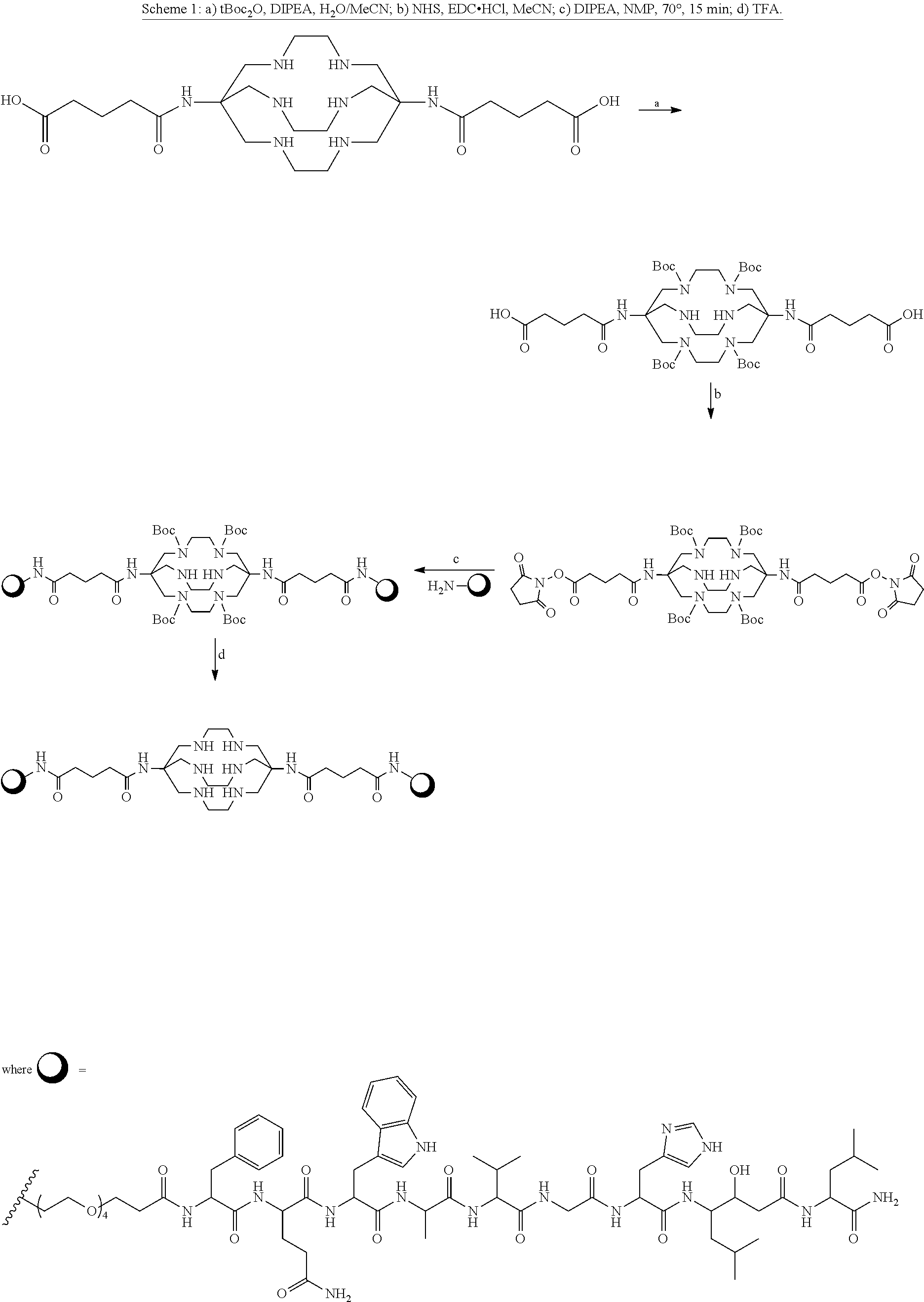

Accordingly, the present invention provides a process for producing a compound of Formula (I), the method comprising the step of coupling a compound of the Formula (II), or a salt, complex, isomer or solvate thereof,

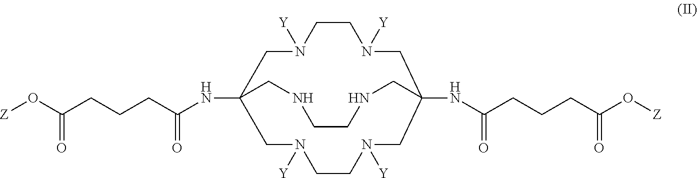

wherein Y is a nitrogen protecting group and Z is an oxygen protecting group;
with a compound of Formula (III) or a salt thereof,

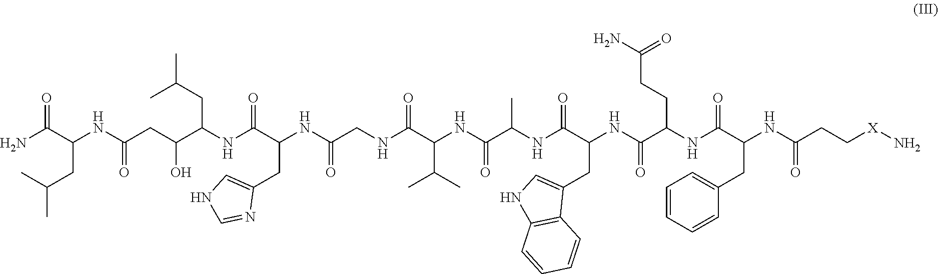

for a time and under conditions to provide a compound of Formula (I).

In an embodiment, the conditions required to produce a compound of Formula (I) are under microwave conditions.

In certain embodiments, the compound of Formula (III) has the structure with stereochemistry specified as follows:

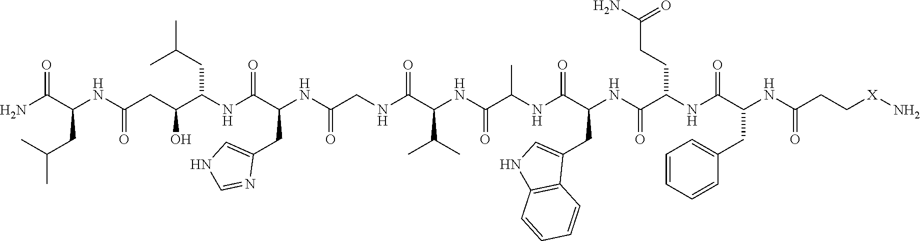

The compound of Formula (II) contains a nitrogen protecting group, i.e. Y. As presently depicted, the compound of Formula (II) may have four nitrogen protecting groups bound to four separate nitrogen atoms in the nitrogen-containing macrocycle. Alternatively, and while not depicted here, the compound of Formula (II) may also have five nitrogen protecting groups bound to five separate nitrogen atoms in the nitrogen-containing macrocycle. In an embodiment, the compound of Formula (II) has four nitrogen protecting groups. In another embodiment, the compound of Formula (II) has five nitrogen protecting groups.

As used herein, the term “nitrogen protecting group” means a group that can prevent the nitrogen moiety reacting during further derivatisation of the protected compound and which can be readily removed when desired. In one embodiment the protecting group is removable in the physiological state by natural metabolic processes and in essence the protected compound is acting as a prodrug for the active unprotected species. Examples of suitable nitrogen protecting groups that may be used include formyl, trityl, phthalimido, acetyl, trichloroacetyl, chloroacetyl, bromoacetyl, iodoacetyl; urethane-type blocking groups such as benzyloxycarbonyl (‘CBz’), 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl (“Boc”), 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxy-carbonyl, cyclo-pentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono)ethoxycarbonyl, 2-(triphenylphosphino)-ethoxycarbonyl, fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalymethoxy carbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobornyloxycarbonyl, 1-piperidyloxycarbonlyl and the like; benzoylmethylsulfono group, 2-nitrophenylsulfenyl, diphenylphosphine oxide, and the like. The actual nitrogen protecting group employed is not critical so long as the derivatised nitrogen group is stable to the condition of subsequent reaction(s) and can be selectively removed as required without substantially disrupting the remainder of the molecule including any other nitrogen protecting group(s). Further examples of these groups are found in: Greene, T. W. and Wuts, P. G. M., Protective Groups in Organic Synthesis, Second edition; Wiley-Interscience: 1991; Chapter 7; McOmie, J. F. W. (ed.), Protective Groups in Organic Chemistry, Plenum Press, 1973; and Kocienski, P. J., Protecting Groups, Second Edition, Thieme Medical Pub., 2000.

The compound of Formula (II) also contains an oxygen protecting group, i.e. Z in the compound of Formula (II).

As used herein, the term “oxygen protecting group” means a group that can prevent the oxygen moiety reacting during further derivatisation of the protected compound and which can be readily removed when desired. In one embodiment the protecting group is removable in the physiological state by natural metabolic processes. Examples of oxygen protecting groups include acyl groups (such as acetyl), ethers (such as methoxy methyl ether (MOM), β-methoxy ethoxy methyl ether (MEM), p-methoxy benzyl ether (PMB), methylthio methyl ether, pivaloyl (Piv), tetrahydropyran (THP)), N-hydroxysuccinimide (NHS) and silyl ethers (such as trimethylsilyl (TMS) tert-butyl dimethyl silyl (TBDMS) and triisopropylsilyl (TIPS).

In an embodiment, the nitrogen protecting group in the compound of Formula (II), i.e. Y is a t-butoxycarbonyl (i.e. Boc) group. In an embodiment, the compound of Formula (II) has four Boc groups. In an embodiment, the compound of Formula (II) has five Boc groups.

In an embodiment, the oxygen protecting group in the compound of Formula (II), i.e. Z, is a N-hydroxysuccinimide (NHS) group.

The compound of Formula (III) contains the bombesin-like peptide attached to the linker comprising a PEG group, where the part of the compound of Formula (III) that is coupled to the compound of Formula (II) is an amine.

The method for the coupling of a compound of Formula (II) with a compound of Formula (III) under microwave conditions as disclosed herein may be performed in the presence of one or more bases. In an embodiment, the method is performed in the presence of one base. In another embodiment, the method is performed in the presence of more than one base.

Examples of bases suitable for use in the coupling of a compound of Formula (II) with a compound of Formula (III) include diisopropylethyl amine (iPr₂EtN, DIPEA) and N-methyl-2-pyrrolidinone (NMP). Non-nucleophilic, organic soluble bases such as. Et₃N, DBU may also be suitable.

Suitable solvents incudes NMP.

In an embodiment, the method for the coupling of a compound of Formula (II) with a compound of Formula (III) under microwave conditions is performed in the presence of DIPEA. In another embodiment, the method is performed in the presence of NMP. In a further embodiment, the method is performed in the presence of both DIPEA and NMP.

The method for coupling of a compound of Formula (II) with a compound of Formula (III) under microwave conditions may be performed at a number of suitable temperatures. A suitable temperature may depend on the exact nature of the compounds of Formulae (II) and (III) and the one or more bases used. For example, the microwave conditions may occur at room temperature or at an elevated temperature.

The time for which a compounds of Formulae (II) and (III) are subjected to microwave conditions may also vary, depending on the exact nature of the compounds and the presence of the one or more bases used.

Upon completion of the method for coupling a compound of Formula (II) to a compound of Formula (III), the protective groups may be removed using techniques well known in the art.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### Synthesis of Compounds of the Invention

The agents of the various embodiments may be prepared using the reaction routes and synthesis schemes as described below, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3^{rd} Edition, John Wiley & Sons, 1991. Alternatively, other reactions disclosed herein or known in the art will be recognized as having applicability for preparing other compounds of the various embodiments. Reagents useful for synthesizing compounds may be obtained or prepared according to techniques known in the art.

### Instrumentation

Mass spectra were collected using a Thermo Scientific Exactive Plus OrbiTrap LC/MS (Thermo Fisher Scientific, Massachusetts, USA) and calibrated to internal references.

NMR spectra were recorded on an Agilent MR400 NMR (California, USA) (¹H at 400 MHz) at 297 K and referenced in internal solvent residue.

Analytical RP-HPLC traces were recorded using an Agilent 1200 HPLC system equipped with an Alltech Hypersil BDS C18 analytical HPLC column (4.6×150 mm, 5 μm) with a flow rate of 1 mL min⁻¹, with UV absorbance being recorded at 214 and 254 nm. Retention times (Rₜ/min) were recorded using a gradient elution of 5-100% B in A (A=0.1% TFA, B=MeCN with 0.1% TFA) over 30 min.

Semi-preparative HPLC was performed on an Agilent 1200 HPLC System using a buffer of A=0.1% TFA and B=0.1% TFA in MeCN with UV detection at 214 nm.

Microwave synthesis was performed using a Biotage (Uppsala, Sweden) Initator+ microwave system.

⁶⁴Cu was obtained from SAHMRI (Adelaide, SA) and provided as a solution in HCl (1 GBq, 100 μL, 0.1 M HCl).

### Bombesin-Like Peptide-PEG

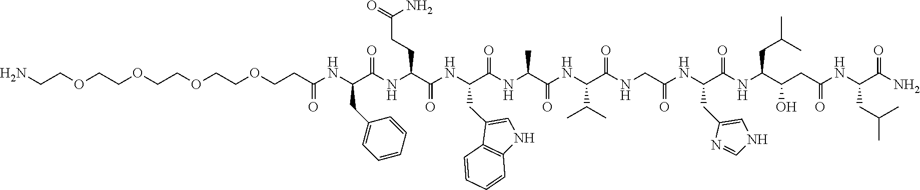 [SMILES: CC(C)C[C@H](NC(=O)C[C@H](O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CCOCCOCCOCCOCCN)C(C)C)C(N)=O]

The peptide was synthesised using an automated, microwave assisted peptide synthesiser (Liberty Blue, CEM, NC, USA) using standard Fmoc-SPPS techniques utilising HATU and DIPEA as coupling reagents on a Rink Amide solid support (125 mg, 0.8 mmol/g, 0.1 mmol. The crude peptide was cleaved from the solid support using TFA/TIPS/H₂O (95:2.5:2.5) before being evaporated to dryness under a stream of N₂ gas. The resulting residue was then dissolved in 30% MeCN in water and purified by semi-preperative HPLC (30% to 100% B in A over 70 min, Phenomonex Luna C18, λ=254 nm). Fractions containing the desired product were collated and lyophilised to yield a colourless powder (26 mg, 0.002 mmol, 2% yield) OTOF/MS [C₆₆H₁₀₁N₁₅O₁₆+2H]²⁺ m/z 680.886 (experimental), 680.885 (calc'd); [C₆₆H₁₀₁N₁₅O₁₆+3H]³⁺ m/z 454.259 (experimental), 454.259 (calc'd). RP-HPLC Rt=11.0 min.

### (tBoc)₄₋₅sar(NHS)₂

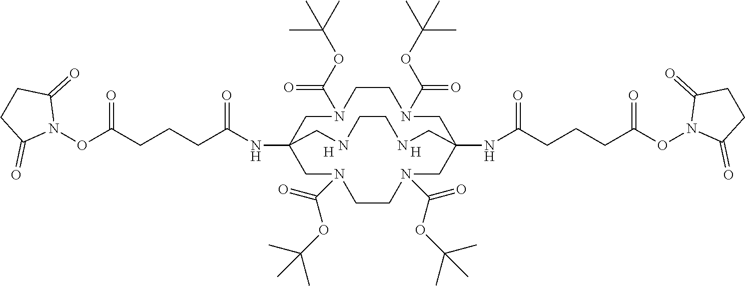

A solution of sar(CO₂H)₂·xHCl (446 mg, 0.82 mmol if x=0) in H₂O/MeCN (1:1, 30 mL) was treated with tBoc₂O (1.02 g, 4.67 mmol) and DIPEA (1 mL, 5.74 mmol) and stirred for 16 h at room temperature. The solvent was then removed under reduced pressure and the resultant residue was suspended in MeCN (40 mL) and treated with EDC-HCl (501 mg, 2.61 mmol) and N-hydroxysuccinamide (300 mg, 2.61), resulting in the dissolution of the suspension. The solution was then evaporated to dryness under reduced pressure and the resulting residue was extracted with CHCl₃ (80 mL), washed with water (3×40 mL) followed by brine (40 mL). The organic extract was then dried over Na₂SO₄, filtered, and evaporated to dryness under reduced pressure to yield a pink-hued residue. The residue was purified by column chromatography (SiO₂, 70 mL, 5% MeOH in CH₂Cl₂, Rf=0.54) to yield a mixture of both (tBu)₄sar(NHS)₂ and (tBu)₅sar(NHS)₂ (136 mg). OTOF/MS [C₅₂H₈₄N₁₀O₁₈+H]⁺ m/z 1137.60 (experimental), 1137.60 (calc'd); [C₅₇H₉₂N₁₀O₂₀+H]⁺ m/z 1237.66 (experimental), 1237.66 (calc'd).

### (tBoc)₄sar(bombesin)₂ and (tBoc)₅sar(bombesin)₂

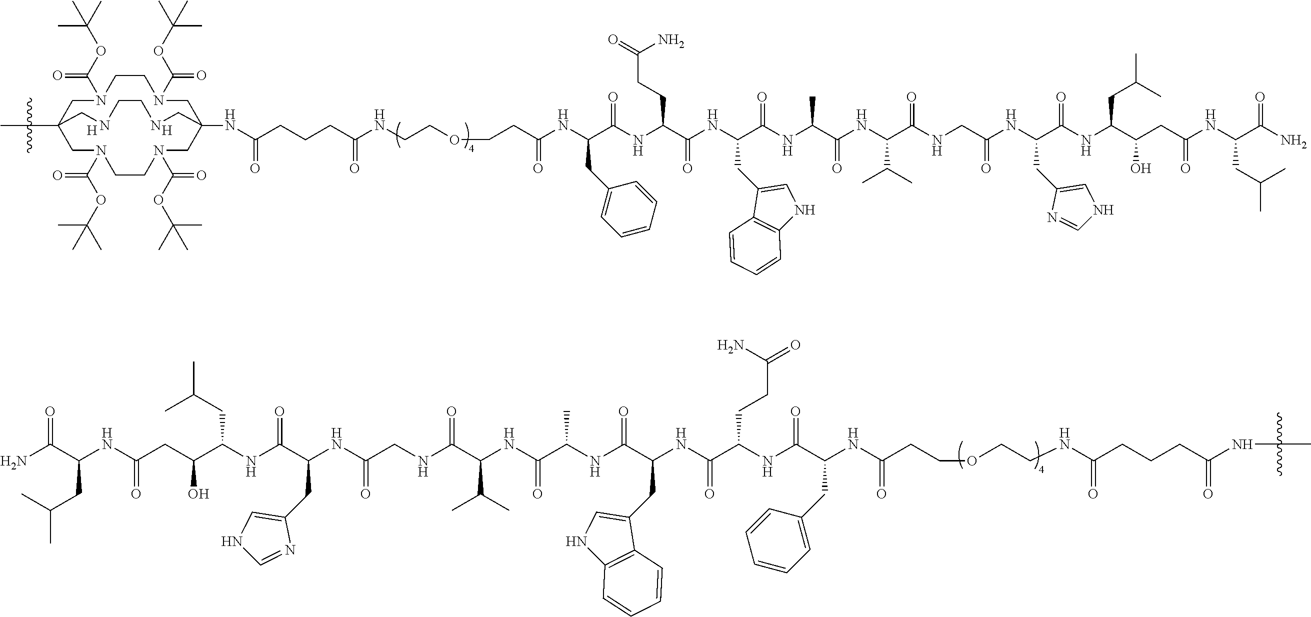

A solution of (tBoc)₄₋₅sar(NHS)₂ (3.5 mg, 3.1 μmol), the bombesin-like peptide-PEG fragment (8.4 mg, 6.2 μmol) and DIPEA (0.1 mL) in N-methylpyrrolidinone (1 mL) was heated by microwave irradiation at 70° C. for 25 min. The resulting solution was then diluted with diethylether, precipitating a colourless solid that was isolated by centrifugation and decantation of the supernatant. The resulting solid was then dissolved in 20% MeCN in water and purified by semi-preparative HPLC (20-100% B in A, 60 min). Fractions containing the desired products were collated and lyophilised to yield flocculent, colourless powders. (tBoc)₄sar(bombesin)₂ (0.9 mg, 0.25 μmol). OTOF/MS: [C₁₇₆H₂₇₆N₃₈O₄₄+3H]³⁺ m/z 1210.361 (experimental), 1210.026 (calculated). RP-HPLC: Rt=14.7 min. (tBoc)₅sar(bombesin)₂ (1.3 mg, 3.5 μmol). OTOF/MS: [C₁₈₁H₂₈₄N₃₈O₄₆+3H]³⁺ m/z 1243.712 (experimental), 1243.377 (calculated). RP-HPLC: Rt=16.3 min.

### Sar(bombesin)₂ (Formula Ia)

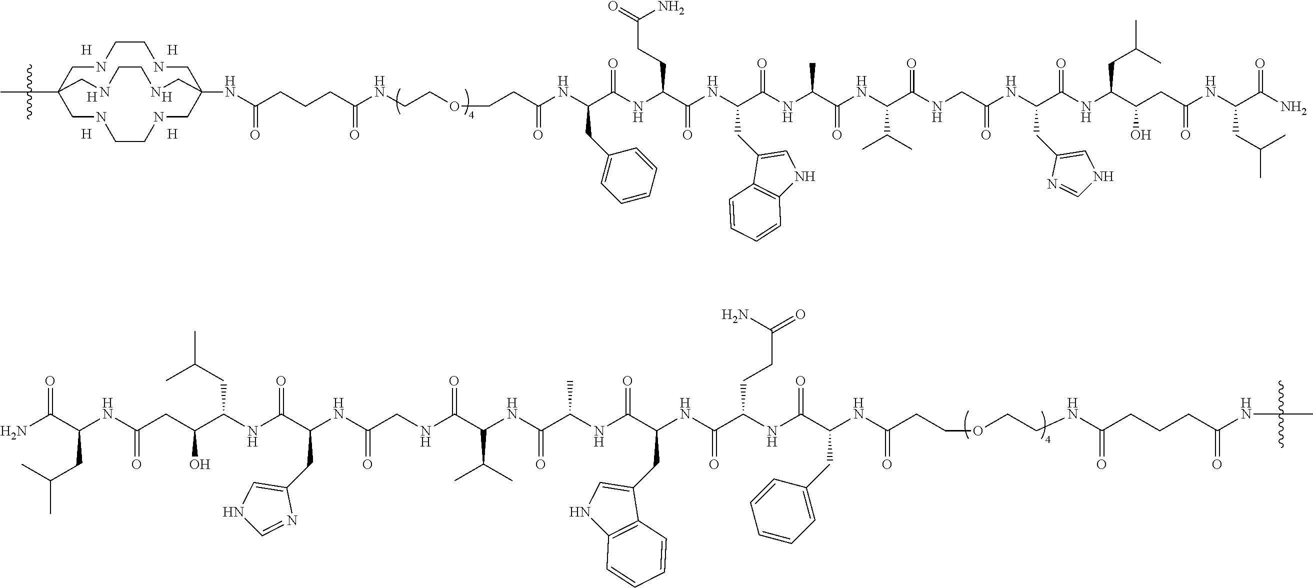

A mixture of (tBoc)₄sar(bombesin)₂ (1 mg, 0.88 0.9 μmol) or (tBoc)₅sar(bombesin)₂ (1.3 mg, 1.1 μmol) in TFA (1 mL) was incubated at room temperature for 1 h before being evaporated to dryness under a stream of N₂ gas. The resulting residues were dissolved in MeCN/H₂O (10%, 1 mL). and purified by semi-preperative HPLC (20-100% B in A, 70 min). Fractions containing the desired product were collated and lyophilised to yield sar(bombesin)₂ (0.2 mg, 0.06 μmol and 0.5 mg, 0.15 μmol respectively). OTOF/MS: [C₁₅₆H₂₄₄N₃₈O₃₆+3H]³⁺ m/z 1076.624 (experimental), 1076.623 (calc'd); [C₁₅₆H₂₄₄N₃₈O₃₆+4H]⁴⁺ m/z 807.721 (experimental), 807.719 (calc'd). RP-HPLC Rt: 11.9 min.

### Radiochemistry

Sar-bombesin₂ was prepared as a theoretical 0.5 μg/μL solution in 50:50 ethanol:water. Phosphate buffer (0.1 M, pH 6.2) was prepared from sodium phosphate in MilliQ water. ⁶⁴CuCl₂ was supplied by SAHMRI. Sodium phosphate buffer (32 μL, 0.1 M, pH 6.2) was added to an Eppendorf tube, followed by an aliquot of Sar-bombesin₂ (2 μL, 1 μg) followed by an aliquot of ⁶⁴Cu (10 MBq, 7 μL, 0.1 M HCl). The pH of the resultant solution was found to be about 6. The mixture was then incubated for 15 minutes before 50 μL of the mixture was taken, diluted in EtOH (100 μL) and analysed by HPLC.

### Radiolabelling and TLC Analysis of Constructs

All constructs were incubated with ⁶⁴Cu at an excess of compound (in 50% EtOH in Na₂PO₄ buffer (0.1 M, pH 6.2) for 15-60 minutes.

Samples of each solution were taken and mixed 1:1 with 50 mM EDTA. 5 μL of each solution was spotted on TLC paper (Agilent iTLC-SG Glass microfiber chromatography paper impregnated with silica gel) and run with 50:50 H₂O:ethanol. Plates were then imaged on an Eckert & Ziegler Mini-Scan and Flow-Count iTLC Reader. HPLC was also performed on samples for the first radiolabelling to validate the TLC results. All samples used for in vivo imaging were >95% labelling.

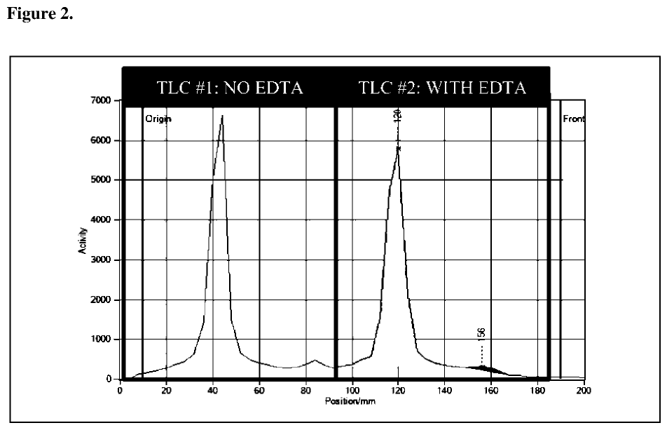

Control experiments were conducted to monitor the elution behaviour of free ⁶⁴Cu and ⁶⁴Cu bound to EDTA for quality control, as well as each sample also run with EDTA to check for radiopurity. A representative radioTLC image showing that all ⁶⁴Cu was bound to the dendrimers can be found in FIG. 2. Results indicated that 100% of the compound was labelled and there was no unbound ⁶⁴Cu in the solution.

### Cell Binding Studies

PC3 cells were seeded at a density of 5×10⁴ cells per well in 24-well plates and incubated overnight with medium (RPMI 160 containing 10% fetal bovine serum and 1% streptomycin-penicillin). Approximately 200 kBq of radio-ligand was added to the medium, and the cells were incubated (in triplicate) for 15, 30, and 60 min at 25° C. At each time point, internalization was stopped by removing the medium and washing the cells twice with ice-cold PBS (pH 7.4, 0.5 mL). To remove the receptor-bound radioligand, an acid wash was carried out twice with ice-cold glycine buffer (0.1 M, pH 3.0, 1 mL) for 5 minutes. Cells were solubilized with NaOH (1 N, 2 mL) and the internalized fractions collected. The radioactivity of the supernatant, receptor-bound and internalized fractions were measured in a gamma-counter. Gamma counts were decay-corrected and converted to Becquerels, and the receptor-bound and internalized fractions represented as a percentage of applied activity per 10⁵ cells.

[⁶⁴Cu]Formula (Ia) showed high total cellular binding over 60 minutes, peaking at 15 minutes (˜2.5%), compared to a maximum cellular binding of 30 minutes (˜1.5%) for the monomer [⁶⁴Cu]Formula (Ib). Total binding decreasing steadily at later time points. An observed decrease in total cellular binding after 30 minutes may have been due to continuous internalisation of the compounds which had reached a steady rate.

### In Vivo Imaging Analysis

### Animals

Healthy male Balb/c nude mice (˜18 g) from 8 weeks old were obtained from the ARC and used for this study. Mice were imported into the CAI animal holding facility and monitored for 1 week prior to the study in order to acclimatize to the environment prior to injection of cells. All animals were provided with free access to food and water before and during the imaging experiments which were approved by the University of Queensland Animal Ethics Committee (Approval #AIBN/CAI/105/19/ARC/NHMRC).

### Tumour Initiation and Growth

8-week-old male Balb/c nude mice were injected (27G needle) subcutaneously with PC3 (2×106) cells in 50 μL of 50:50 matrigel and cells in phosphate buffered saline into the right flank of each mouse. There was no evidence of ulceration at the time of dosing; the animals were closely monitored and remained in good condition apart from the growth of tumours. The tumour growth was observed to be in line with expected timelines and good tumours were ultimately observed >80% of inoculated animals. Labelled peptides were injected via the tail vein (29G needle; ˜2-3 MBq) and then mice were imaged using the Siemens Inveon PET-CT instrument at the various time points.

### Imaging Protocol

Mice were anaesthetised with isoflurane (IsoFlo, Abbott Laboratories) at a dose of 2% in a closed anaesthetic induction chamber. Mice were monitored using ocular and pedal reflexes to ensure deep anaesthesia. Once the mouse was deeply anesthetised, it was placed on an appropriate animal bed, where the anaesthetic air mixture (1%) was delivered to its nose and mouth through a nose cone. Physiological monitoring (respiratory using a sensor probe) was achieved throughout all experiments using an animal monitoring system (the BioVet™ system, m2m Imaging, Australia). Images were acquired using a Siemens Inveon PET-CT scanner following tail vein intravenous injection of the test articles.

The injection syringe was filled with the radioisotope solution (approximately 150 μL) and the activity in the syringe was measured using a dose calibrator (Capintec CRC-25) with a calibration factor of 35. The activity left in the syringe after the tail vein injection was measured using the same dose calibrator and the total volume injected in each mouse was calculated.

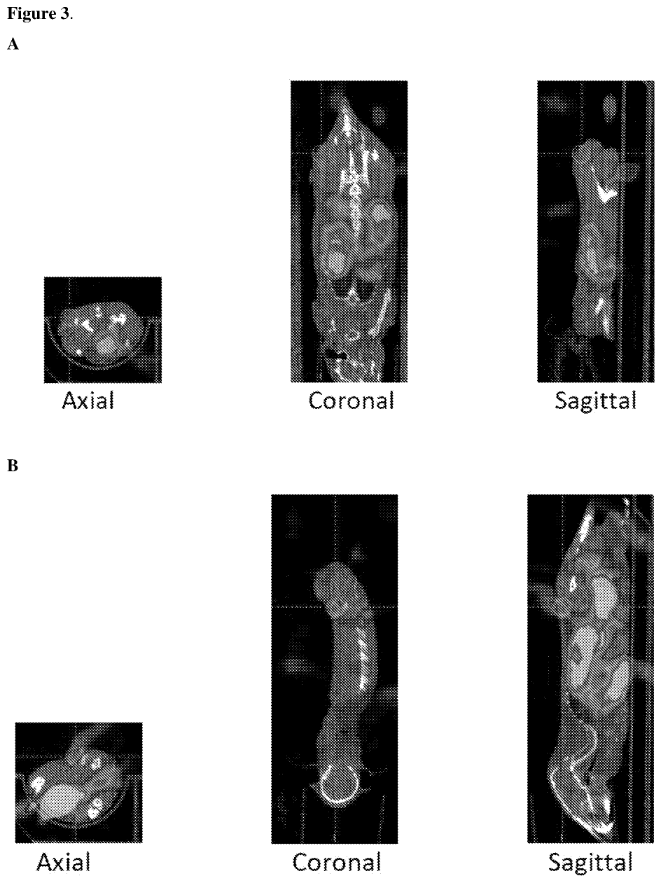

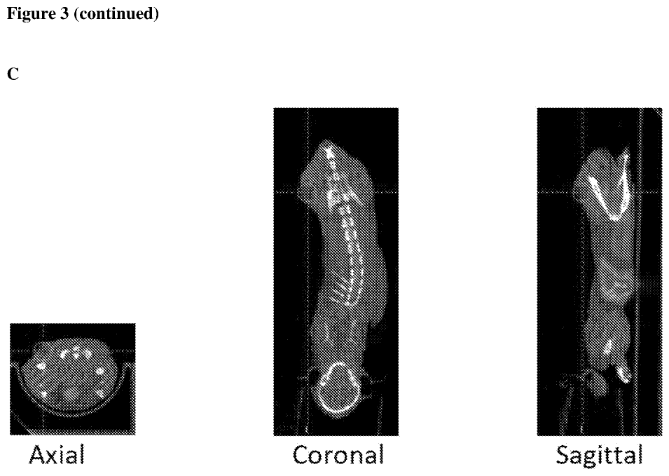

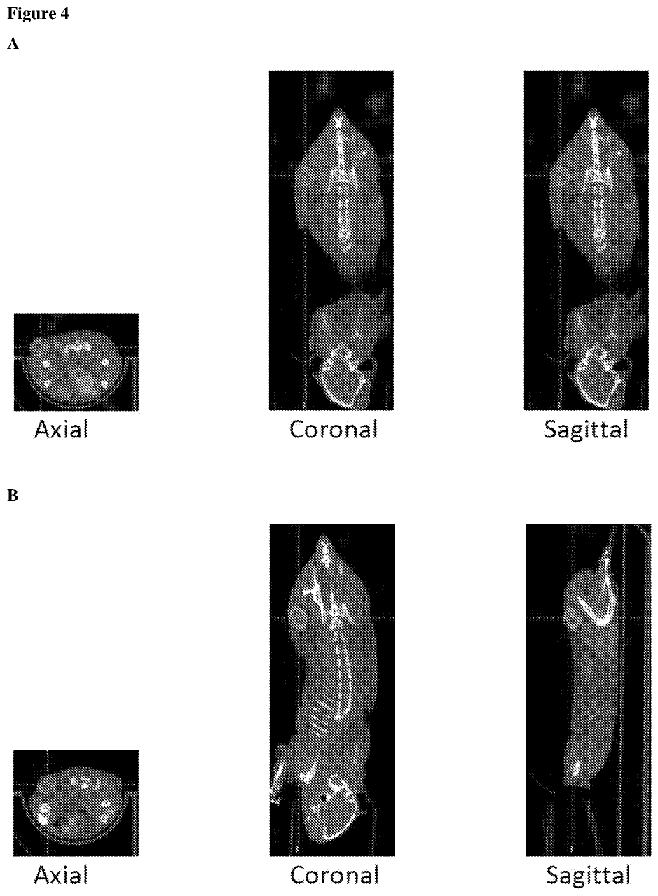

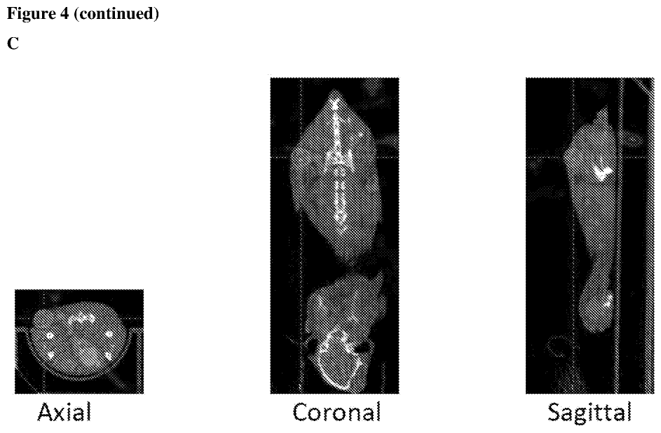

Calibration of the PET/CT scanner was performed with an in-house manufactured phantom containing a known activity of ⁶⁸Ge solution as a radiation source. The mice were positioned on the scanner bed (n=4 per scan using a bed developed in-house) and micro-CT scans were acquired for anatomical co-registration. The CT images of the mice were acquired through an X-ray source with the voltage set to 80 kV and the current set to 500 PA. The scans were performed using 3600 rotation with 120 rotation steps with a low magnification and a binning factor of four. The exposure time was 230 ms with an effective pixel size of 106 μm. The total CT scanning process took approximately 15 minutes. The CT images were reconstructed using Feldkamp reconstruction software (Siemens). Following CT imaging, PET scans were acquired at, 1 hour, 4 hours and 24 hours after injection of the radiotracer (see FIGS. 3A-C and 4A-C) using 30-90-minute static acquisitions. All images were static acquisitions. The PET Images were reconstructed using an ordered-subset expectation maximization (OSEM2D) algorithm and analysed using the Inveon Research Workplace software (IRW 4.1) (Siemens) which allows fusion of CT and PET images and definition of regions of interest (ROIs). CT and PET datasets of each individual animal were aligned using IRW software (Siemens) to ensure good overlap of the organs of interest. Three dimensional ROIs were placed within the whole body, as well as all the organs of interest, such as heart, kidney, lungs, bladder, liver, spleen, pancreas and tumour, using morphologic CT information to delineate organs. Activity per voxel was converted to nCi/cm³ using a conversion factor obtained by scanning a cylindrical phantom filled with a known activity of ⁶⁴Cu to account for PET scanner efficiency. Activity concentrations were then expressed as percent of the decay-corrected injected activity per cm³ of tissue that can be approximate as percentage injected dose/g (% ID/g).

### Injected Dose (% ID/g) in Excised Organs

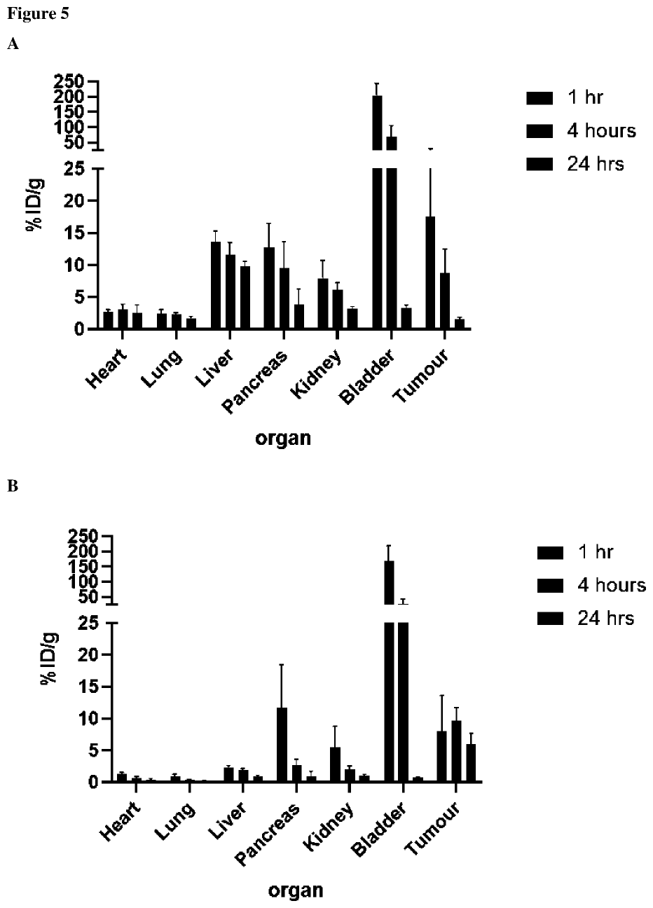

Organs were excised at 24 hours, with the percentage of injected dose of the administered compound determined by ex vivo gamma counting (Table 1, FIG. 5). Values are averages across 4 mice.

TABLE 1
Percentage of injected dose of [⁶⁴Cu]Formula (Ia)
and [⁶⁴Cu]Formula(Ib) in organs after 24 hours, as
determined by ex vivo gamma counting.

| | [⁶⁴Cu]Formula (Ib) | [⁶⁴Cu]Formula (Ia) |
|---|---|---|
| | (% ID/g) | (% ID/g) |
| Tumour | 4.09 ± 0.52 | 2.13 ± 0.86 |
| Liver | 1.52 ± 0.18 | 16.80 ± 1.55 |
| Spleen | 0.38 ± 0.06 | 1.47 ± 0.20 |
| Kidneys | 3.55 ± 0.79 | 6.81 ± 0.67 |
| Heart | 0.24 ± 0.02 | 0.21 ± 0.03 |
| Lungs | 0.31 ± 0.08 | 0.36 ± 0.12 |
| Blood | 0.13 ± 0.04 | 0.10 ± 0.05 |
| Pancreas | 0.23 ± 0.02 | 0.84 ± 0.06 |
| GI Tract | 0.30 ± 0.07 | 0.44 ± 0.04 |
| Tail | 0.56 ± 0.87 | 0.28 0.05 |

### Post-Imaging Analysis of Tumour Uptake

Variability in quantitation between in vivo and ex vivo measurements arises due to region of interest (ROI) and background signal for the in vivo plots. For the systems under study, all compounds showed some degree of tumour accumulation. High pancreatic uptake was observed for the PC3 tumours. It was observed that compounds with two bombesin groups (i.e. the dimer), showed high accumulation shortly after injection (i.e. 1 hr) whereas accumulation of the monomer was more gradual and showed higher accumulation at later time points.

### Preparation of a Single Dose Formulation Comprising ⁶⁴Cu Complexed to a Compound of Formula (Ib)

Sodium gentisate (1 mg) is dissolved in 0.1 M sodium phosphate buffer solution (2 mL) to provide a first solution (Solution A). 20 μg of the compound of Formula (Ib) is then dissolved in Solution A (2.0 mL) to provide a Reaction Vial.

The radioactivity of a ⁶⁴Cu chloride solution is measured and the time recorded. The ⁶⁴Cu chloride solution is then added into the Reaction Vial containing the compound of Formula (Ib) in solution and is held at ambient temperature for 25 minutes.

A second solution (Solution B) is prepared by dissolving sodium ascorbate (250 mg) in TraceSELECT water (1.4 mL) and ethanol (0.14 mL).

The contents of the Reaction Vial are then transferred via a sterile filter into a Final Product Vial. Solution B is drawn up (0.5 mL) in a 5 mL syringe and used to rinse out the Reaction Vial. The contents of the Reaction Vial are transferred via the sterile filter into the Final Product Vial before gently homogenising the contents. The activity within the Final Product Vial is assayed and the EOS time and final product volume are recorded.

### Preparation of a 2-3 Patient Dose of ⁶⁴Cu Complexed to a Compound of Formula (Ib)

Sodium gentisate (3 mg) is dissolved in 0.1 M sodium phosphate buffer solution (6 mL) to provide a first solution (Solution A). 60 μg of the compound of Formula (Ib) is then dissolved in Solution A (6 mL) to provide a Reaction Vial.

The radioactivity of a ⁶⁴Cu chloride solution is measured and the time recorded. The ⁶⁴Cu chloride solution is then added into the Reaction Vial containing the compound of Formula (Ib) in solution and is held at ambient temperature for 25 minutes.

A second solution (Solution B) is prepared by dissolving sodium ascorbate (750 mg) in TraceSELECT water (4.2 mL) and ethanol (0.42 mL).

The contents of the Reaction Vial are then transferred via a sterile filter into a Final Product Vial. Solution B is drawn up (1.5 mL) in a 5 mL syringe and used to rinse out the Reaction Vial. The contents of the Reaction Vial are transferred via the sterile filter into the Final Product Vial before gently homogenising the contents. The activity within the Final Product Vial is assayed and the EOS time and final product volume are recorded.

Table 2 below reproduces a quality control test summary for a 3 dose formulation comprising [⁶⁴Cu]Formula (Ib) prepared according to the above method.

TABLE 2
Quality control test summary for a 3 dose aqueous
formulation comprising [⁶⁴Cu]Formula (Ib)

| | | Batch QC | | |
|---|---|---|---|---|
| QC Test Performed | Acceptance Criteria | Results | Pass | Fail |
| Appearance/Visible | Final product is clear, | Clear ✓ | ✓ | |
| particles in injection | colourless to pale yellow, | Colourless to | | |
| | and free of visible | pale yellow ✓ | | |
| | particulates | Particle-free ✓ | | |
| pH by probe | 4.0-8.0 | 6.83 | ✓ | |
| Free [⁶⁴Cu]Cu²⁺ by TLC | Free [⁶⁴Cu]Cu²⁺ ≤5% | 0.17% | ✓ | |
| Radiochemical purity by | RCP by radio-HPLC ≥90% | 96.48% | ✓ | |
| radio-HPLC | | | | |
| Radiochemical identity by | RT of [64Cu]Formula (Ib) | 0.18% | ✓ | |
| radio-HPLC | sample is within ±5% of | | | |
| | RT of reference std. | | | |
| Chemical identity and | Sodium Gentisate: | 214 μg/mL | ✓ | |
| content by UV-HPLC | Formula (Ib): | 0.00345 μg/mL | | |
| | [64Cu]Formula (Ib): | 0.00474 μg/mL | | |
| Radionuclide identity by | Peaks should be identified | 510.97 | ✓ | |
| Gamma Spec | at 511 and 1346 keV | 1345.90 | | |
| Bacterial endotoxin | ≤11.6 EU/mL | <2.00 | ✓ | |
| content by endosafe-PTS | | | | |
| Radioactive concentration | ≤10.8 mCi/mL at EOS | 0.24 | ✓ | |
| by dose calibrator | | | | |
| Sterility by direct | No growth after 14 days of | Pass | ✓ | |
| inoculation | incubation | | | |

### Preparation of a 4-5 Patient Dose of ⁶⁴Cu Complexed to a Compound of Formula (Ib)

Sodium gentisate (5 mg) is dissolved in 0.1 M sodium phosphate buffer solution (10 mL) to provide a first solution (Solution A). 100 μg of the compound of Formula (Ib) is then dissolved in Solution A (10 mL) to provide a Reaction Vial.

The radioactivity of a ⁶⁴Cu chloride solution is measured and the time recorded. The ⁶⁴Cu chloride solution (0.2 mL) is then added into the Reaction Vial containing the compound of Formula (Ib) in solution and is held at ambient temperature for 25 minutes.

A second solution (Solution B) is prepared by dissolving sodium ascorbate (1.25 g) in TraceSELECT water (7 mL) and ethanol (0.7 mL).

The contents of the Reaction Vial are then transferred via a sterile filter into a Final Product Vial. Solution B is drawn up (3 mL) in a 5 mL syringe and used to rinse out the Reaction Vial. The contents of the Reaction Vial are transferred via the sterile filter into the Final Product Vial before gently homogenising the contents. The activity within the Final Product Vial is assayed and the EOS time and final product volume are recorded.

Table 3 below reproduces a quality control test summary for a 5 dose formulation comprising [⁶⁴Cu]Formula (Ib) prepared according to the above method.

TABLE 3
Quality control test summary for a 5 dose aqueous
formulation comprising [⁶⁴Cu]Formula (Ib)

| | | Batch QC | | |
|---|---|---|---|---|
| QC Test Performed | Acceptance Criteria | Results | Pass | Fail |
| Appearance/Visible | Final product is clear, | Clear ✓ | ✓ | |
| particles in injection | colourless to pale yellow, | Colourless to | | |
| | and free of visible | pale yellow ✓ | | |
| | particulates | Particle-free ✓ | | |
| pH by probe | 4.0-8.0 | 6.85 | ✓ | |
| Free [⁶⁴Cu]Cu²⁺ by TLC | Free [⁶⁴Cu]Cu²⁺ ≤5% | 0.06% | ✓ | |
| Radiochemical purity by | RCP by radio-HPLC ≥90% | 97.66% | ✓ | |
| radio-HPLC | | | | |
| Radiochemical identity by | RT of [64Cu]Formula (Ib) | 0.46% | ✓ | |
| radio-HPLC | sample is within ±5% of | | | |
| | RT of reference std. | | | |
| Chemical identity and | Sodium Gentisate: | 200 μg/mL | ✓ | |
| content by UV-HPLC | Formula (Ib): | 244 μg/mL | | |
| | [64Cu]Formula (Ib): | 278 μg/mL | | |
| Radionuclide identity by | Peaks should be identified | Pass | ✓ | |
| Gamma Spec | at 511 and 1346 keV | | | |
| Bacterial endotoxin | ≤11.6 EU/mL | 1.12 | ✓ | |
| content by endosafe-PTS | | | | |
| Radioactive concentration | ≤10.8 mCi/mL at EOS | 7.52 | ✓ | |
| by dose calibrator | | | | |
| Sterility by direct | No growth after 14 days of | Pass | ✓ | |
| inoculation | incubation | | | |

### Preparation of a 10 Patient Dose of ⁶⁴Cu Complexed to a Compound of Formula (Ib)

Sodium gentisate (10 mg) is dissolved in 0.1 M sodium phosphate buffer solution (20 mL) to provide a first solution (Solution A). 200 μg of the compound of Formula (Ib) is then dissolved in Solution A (14 mL) to provide a Reaction Vial.

The radioactivity of a ⁶⁴Cu chloride solution is measured and the time recorded. The ⁶⁴Cu chloride solution is then added into the Reaction Vial containing the compound of Formula (Ib) in solution and is held at ambient temperature for 25 minutes.

A second solution (Solution B) is prepared by dissolving sodium ascorbate (2.5 g) in TraceSELECT water (14 mL) and ethanol (1.4 mL).

The contents of the Reaction Vial are then transferred via a sterile filter into a Final Product Vial. Solution B is drawn up (15.0 mL) in a 20 mL syringe and used to rinse out the Reaction Vial. The contents of the Reaction Vial are transferred via the sterile filter into the Final Product Vial before gently homogenising the contents. The activity within the Final Product Vial is assayed and the EOS time and final product volume are recorded.

### Stability of Formulations of the Invention

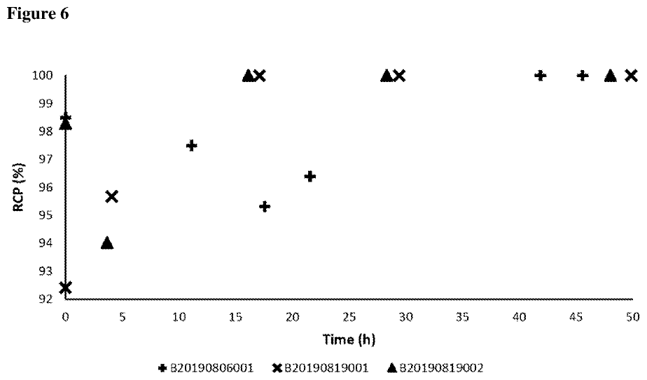

Product stability was monitored up to 48 hours post End of Synthesis (EOS) for 3 validation batches of [⁶⁴Cu]Formula (Ib) in an aqueous formulation prepared as outlined above. Due to time restrictions, identical stability sampling time points were not always achievable. Over the period of the test, the radiochemical purity (RPC) did not fall below 92.4% for each of the batches. As illustrated in FIG. 6, the RCP appears quantitative when analysed beyond the 25 hour time point. This is attributed to radioactive decay reducing the peak area of radiolytic impurities to below detectable limits, rather than a true increase in the radiochemical purity.

These results indicate aqueous formulations of [⁶⁴Cu]Formula (Ib) prepared by the processes described herein meet the acceptance criteria and conform to the specifications required for injectable formulations.

1. A compound of Formula (I), or a salt, complex, isomer, solvate, prodrug or protected form thereof: 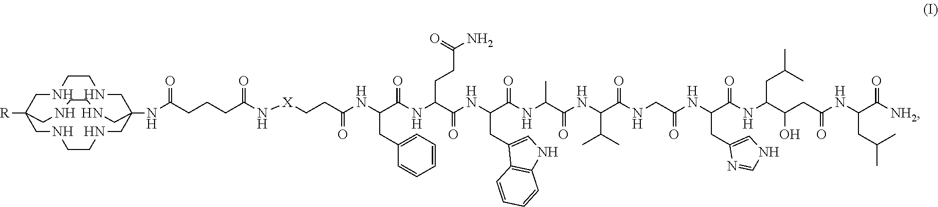
wherein:
X is 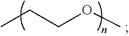
n is an integer from 1 to 10; and
R is selected from the group consisting of H, OH, halogen, cyano, NO₂, optionally substituted C₁-C₁₂ alkyl, optionally substituted amino, optionally substituted amide, optionally substituted aryl and formula (A):

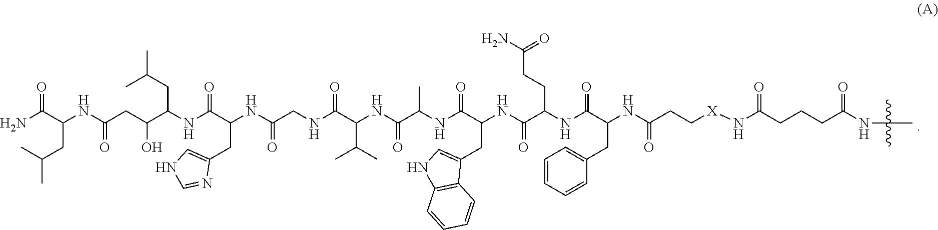

2. A compound according to claim 1, or a salt, complex, isomer, solvate or prodrug thereof, wherein R is an optionally substituted C₁-C₁₂ alkyl group or an optionally substituted amide.

3. (canceled)

4. A compound according to claim 1, or a salt, complex, isomer, solvate or prodrug thereof, wherein R is formula (A):

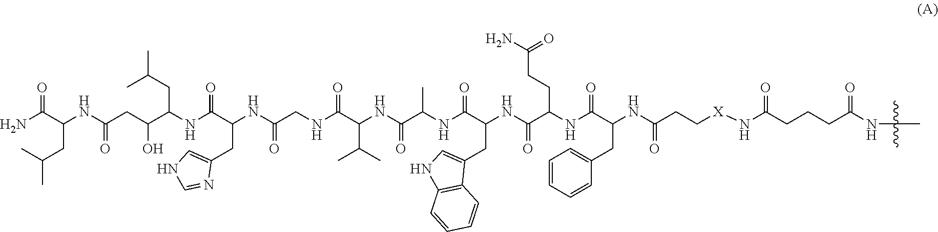

5. A compound according to claim 1, or a salt, complex, isomer, solvate or prodrug thereof, wherein X is 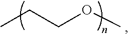
and n is an integer from 1 to 10.

6. (canceled)

7. A compound according to claim 1, or a salt, complex, isomer, solvate or prodrug thereof, having the following structure:

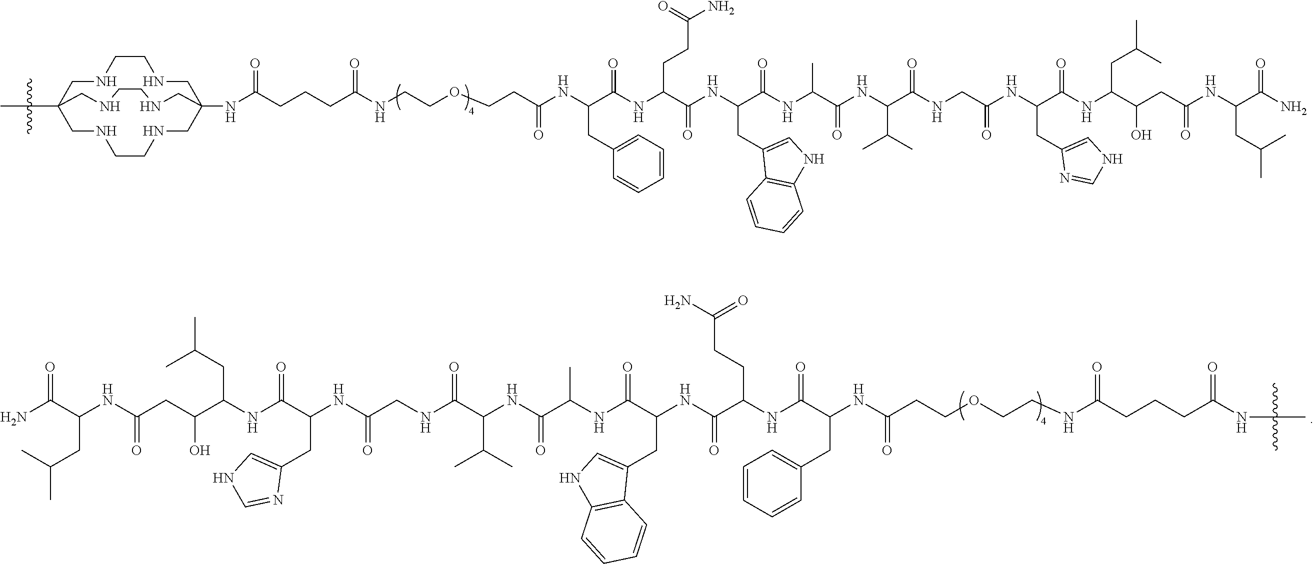

8. A compound according to claim 1, wherein the compound is coordinated with a metal ion.

9. (canceled)

10. A compound according to claim 8, where in the metal ion is a radionuclide of Cu.

11. A compound according to claim 10, wherein the metal ion is a radionuclide selected from the group consisting of ⁶⁰Cu, ⁶¹Cu, ⁶²Cu, ⁶⁴Cu and ⁶⁷Cu.

12. A composition comprising a compound according to claim 1, and one or more pharmaceutically acceptable excipients.

13. A process for producing a compound of Formula (I) as defined in claim 1 or a salt, complex, isomer, solvate, prodrug or protected form thereof,
wherein R is a group of Formula (A): 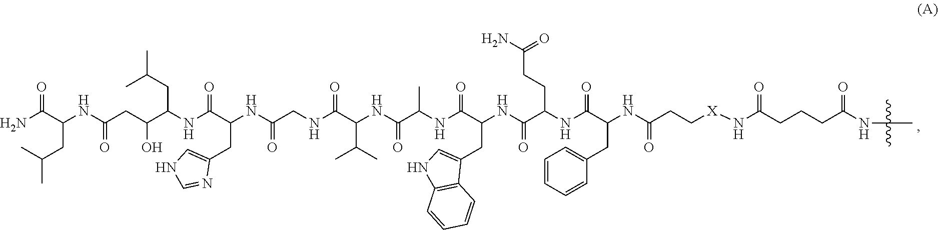
the method comprising coupling a compound of Formula (II), or a salt, complex, isomer or solvate thereof, 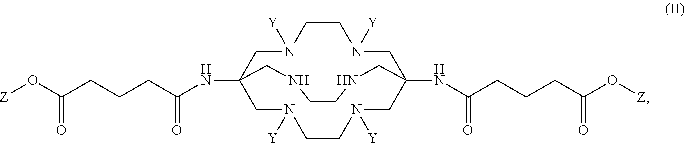
wherein Y is a nitrogen-protecting group and Z is an oxygen-protecting group;
with a compound of Formula (III) or a salt thereof, 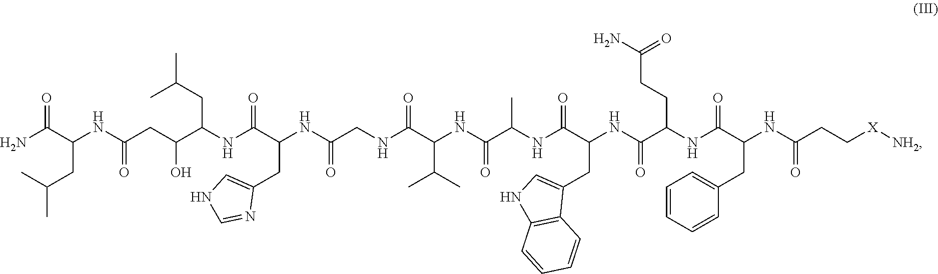
for a time and under conditions to provide the compound of Formula (I).

14. A process according to claim 13, wherein the compound of Formula (II) and the compound of Formula (III) are coupled together under microwave conditions.

15. (canceled)

16. A method of treating a cancer in a subject, the method comprising administering to a subject in need thereof a compound as defined in claim 1.

17. The method of claim 16, wherein the cancer is any malignant cancerous or pre-cancerous cell growth.

18. A method of radioimaging a subject, the method comprising administering to a subject in need thereof a compound as defined in claim 10, wherein the radioimaging is by PET or SPECT.

19. (canceled)

20. An aqueous formulation comprising a compound of Formula (I) as defined in claim 1 or a salt, isomer, solvate, prodrug or protected form thereof, complexed with a radionuclide, a buffer and one or more excipients.

21. The aqueous formulation according to claim 20, wherein the formulation comprises sodium phosphate buffer, gentisic acid, or a salt thereof, ethanol and ascorbic acid, or a salt thereof.

22. (canceled)

23. The aqueous formulation according to claim 20, wherein the radionuclide is selected from the group consisting of ⁶⁰Cu, ⁶¹Cu, ⁶²Cu, ⁶⁴Cu, and ⁶⁷Cu.

24-31. (canceled)

32. A process for preparing an aqueous formulation comprising a compound of Formula (I) as defined in claim 1 or a salt, isomer, solvate, prodrug or protected form thereof complexed with a radionuclide, the method comprising:
i) dissolving a compound of Formula (I), or a salt thereof, in a buffer solution comprising gentisic acid, or a salt thereof;
ii) adding a solution of a radionuclide to the solution of i);
iii) filtering the solution obtained from ii); and
iv) quenching the reaction by addition of aqueous ethanol and ascorbic acid;
to recover an aqueous formulation comprising a compound of Formula (I), or a salt thereof, complexed with a radionuclide.

33-34. (canceled)

35. The process according to claim 32, wherein the radionuclide is selected from the group consisting of ⁶⁰Cu, ⁶¹Cu, ⁶²Cu, ⁶⁴Cu, and, ⁶⁷Cu.

36. (canceled)

37. A kit for making an aqueous formulation comprising a compound of Formula (I) as defined in claim 1, or a salt, isomer, solvate, prodrug or protected form thereof, complexed with a radionuclide, the kit comprising:
a container comprising a lyophilised compound of Formula (I) or a salt, isomer, solvate, prodrug or protected form thereof;
a container comprising a solution of the radionuclide; and
instructions for preparing an aqueous formulation by adding sodium phosphate buffer, ethanol, gentisic acid, or a salt thereof and ascorbic acid, or a salt thereof.

38. (canceled)
